(19) United States
(12) Patent Application Publication
(10) Pub. No.: US 2026/0162833 A1
(43) Pub. Date: Jun. 11, 2026

(54) TECHNOLOGIES FOR HEALTH AND/OR ENVIRONMENTAL EXPOSURE TRACKING AND/OR NOTIFICATION VIA SMARTPHONES AND/OR SOCIAL MEDIA

(71) Applicant: Colgate-Palmolive Company, New York, NY (US)

(72) Inventors: Donghui WU, Bridgewater, NJ (US); Hongbing LI, Skillman, NJ (US)

(73) Assignee: Colgate-Palmolive Company, New York, NY (US)

(21) Appl. No.: 19/413,336

(22) Filed: Dec. 9, 2025

Related U.S. Application Data
(60) Provisional application No. 63/729,598, filed on Dec. 9, 2024.
(60) Provisional application No. 63/729,620, filed on Dec. 9, 2024.

(51) Int. Cl. G16H 50/80; H04W 4/029

(52) U.S. Cl. CPC G16H 50/80; H04W 4/029

(57) ABSTRACT

Systems, devices, and methods for providing health and/or environmental exposure tracking and/or notifications. At least a first sensor may be configured to detect at least one of: a health parameter, or an environmental exposure parameter, corresponding to at least a first user. An electronic device may comprise a transceiver configured to communicate with at least one of: the first sensor, or a second processor. A first processor, the first processor may configure at least one social media application on the electronic device. Processed data corresponding to the health parameter and/or the environmental exposure parameter, may be received from the second processor for presentation via the at least one social media application. The processed data corresponding to the at least one of: the health parameter, or the environmental exposure parameter, may be presented via one or more user-initiated sessions of the at least one social media application.

### CROSS-REFERENCE TO RELATED APPLICATIONS

The present application claims the benefit of priority to U.S. Provisional Patent Application Ser. No. 63/729,598, filed Dec. 9, 2024, and U.S. Provisional Patent Application Ser. No. 63/729,620, filed Dec. 9, 2024, the entireties of which are incorporated herein by reference.

### BACKGROUND

There are various types of ultraviolet (UV) light. UVB light rays are the primary cause of sunburn. UVB exposure may accelerate skin aging, suppress some immune function, and may contribute to the development of skin cancer. UVA light rays, though perhaps less intense than UVB rays, may be 30 to 50 times more prevalent than UVB rays. Prolonged and/or cumulative UVA exposure may damage the collagen fibers in your skin, which contributes to signs of aging, such as wrinkles, age spots, and/or loss of elasticity.

Some potential complications of cumulative sun exposure include skin cancer, including basal cell carcinoma, squamous cell carcinoma, and melanoma; sunburn and blisters; heat rash; premature skin aging, including wrinkles and/or sunspots; retina damage; corneal sunburn and/or inflammation; formation of cataracts, and/or a weakened immune system. More than 90% UV radiation can pass through clouds. Clean snow can reflect up to 80% of UV radiation. UV radiation can increase 4% every 300 meters above sea level. White sand may reflect up to 15% of UV radiation.

Those who work inside buildings, on average, receive 5-10 times less UV radiation annually than those who work outside. At a depth of 0.5 m in water, the UV radiation level is 40% of that at the surface. Sixty percent of the total UV radiation people may be exposed to is received between 10 a.m, and 2 p.m. Shade may decrease UV radiation level by 50% or more.

### BRIEF SUMMARY

A system for providing health and/or environmental exposure tracking and/or notifications may comprise at least a first sensor that may be configured to detect at least one of: a health parameter, or an environmental exposure parameter, corresponding to at least a first user. The system may comprise an electronic device. The electronic device may comprise a memory, a display, and/or a transceiver. The transceiver may be configured to communicate with at least one of: the first sensor, or a second processor. The first processor may be configured to configure at least one social media application on the electronic device. The first processor may be configured to receive, from the second processor, processed data corresponding to the at least one of: the health parameter, or the environmental exposure parameter, for presentation via the at least one social media application configured on the electronic device. The first processor may be configured to present the processed data corresponding to the at least one of: the health parameter, or the environmental exposure parameter, via one or more user-initiated sessions of the at least one social media application.

A system for providing health and/or environmental exposure tracking and/or notifications may comprise a processing device comprising at least a first processor. The first processor may be configured to receive, from a location service, location data corresponding to a location of an electronic device. The first processor may be configured to receive, from an environmental information service, an environmental exposure parameter associated with the location of the electronic device. The first processor may be configured to select at least one social media application configured on the electronic device. The first processor may be configured to generate an exposure data based on the environmental exposure parameter and the location data. The first processor may be configured to process the exposure data for presentation via the at least one social media application. The first processor may be configured to provide, to the electronic device, the processed exposure data for presentation via the at least one social media application.

The electronic device may comprise a memory, a display, and/or a transceiver. The transceiver may be configured to communicate with at least one of: the location service, the environmental information service, or the first processor. The electronic device may comprise a second processor. The second processor configured may be configured to configure the at least one social media application on the electronic device. The second processor may be configured to receive, from the first processor, the processed exposure data for presentation via the at least one social media application. The second processor may be configured to present the processed exposure data for presentation via the at least one social media application via one or more user-initiated sessions of the at least one social media application.

An electronic device for providing health and/or environmental exposure tracking and/or notifications may comprise a memory, a display, and/or a transceiver. The transceiver may be configured to communicate with one or more global position system (GPS) indicator devices and/or to communicate with an environmental information service. The electronic device may comprise a processor. The processor may be configured to determine, from the one or more global position system (GPS) indicator devices, a location of the electronic device. The processor may be configured to determine, from the environmental information service, an ultraviolet (UV) light index based on the location of the electronic device. The processor may be configured to determine, from the one or more global position system (GPS) indicator devices, movement data corresponding to the electronic device. The processor may be configured to determine at least one scene corresponding to the location of the electronic device. The processor may be configured to determine an activity type corresponding to the movement data. The processor may be configured to assign at least one multiplier to the scene and the activity type. The processor may be configured to determine a UV exposure corresponding to the location of the electronic device based, at least in part, on: a multiplied scene, a multiplied activity type, and the UV light index. The processor may be configured to present the UV exposure corresponding to the electronic device via the display of the electronic device.

A method for providing health and/or environmental exposure tracking and/or notifications may comprise detecting, by at least a first sensor, at least one of: a health parameter, or an environmental exposure parameter, corresponding to at least a first user. The method may comprise communicating, by a transceiver, with at least one of: the first sensor, or a second processor. The method may comprise configuring, by a first processor, at least one social media application on an electronic device. The method may comprise receiving, by the first processor from the second processor, processed data corresponding to the at least one of: the health parameter, or the environmental exposure parameter, for presentation via the at least one social media application configured on the electronic device. The method may comprise presenting the processed data corresponding to the at least one of: the health parameter, or the environmental exposure parameter, via one or more user-initiated sessions of the at least one social media application.

A method for providing health and/or environmental exposure tracking and/or notifications may comprise receiving, by a first processor from a location service, location data corresponding to a location of an electronic device. The method may comprise receiving, by the first processor from an environmental information service, an environmental exposure parameter associated with the location of the electronic device. The method may comprise selecting, by the first processor, at least one social media application configured on an electronic device. The method may comprise generating, by the first processor, an exposure data based on the environmental exposure parameter and the location data. The method may comprise processing, by the first processor, the exposure data for presentation via the at least one social media application. The method may comprise providing, by the first processor to the electronic device, the processed exposure data for presentation via the at least one social media application. The method may comprise communicating, by a transceiver, with at least one of: the location service, the environmental information service, or the first processor. The method may comprise configuring, by a second processor, the at least one social media application on the electronic device. The method may comprise receiving, by the second processor from the first processor, the processed exposure data for presentation via the at least one social media application. The method may comprise presenting, by the second processor, the processed exposure data for presentation via the at least one social media application via one or more user-initiated sessions of the at least one social media application.

A method performed by an electronic device for providing health and/or environmental exposure tracking and/or notifications may comprise communicating, by a transceiver, with one or more global position system (GPS) indicator devices. The method may comprise communicating, by the transceiver, with an environmental information service. The method may comprise determining, by a processor from the one or more global position system (GPS) indicator devices, a location of the electronic device. The method may comprise determining, by the processor from the environmental information service, an ultraviolet (UV) light index based on the location of the electronic device. The method may comprise determining, by the processor from the one or more global position system (GPS) indicator devices, movement data corresponding to the electronic device. The method may comprise determining, by the processor, at least one scene corresponding to the location of the electronic device. The method may comprise determining, by the processor, an activity type corresponding to the movement data. The method may comprise assigning, by the processor, at least one multiplier to the scene and the activity type. The method may comprise determining, by the processor, a UV exposure corresponding to the location of the electronic device based, at least in part, on: a multiplied scene, a multiplied activity type, and the UV light index. The method may comprise presenting, by the processor, the UV exposure corresponding to the location of the electronic device via a display of the electronic device.

### BRIEF DESCRIPTION OF THE DRAWINGS

The elements and other features, advantages and disclosures contained herein, and the manner of attaining them, may become apparent and the present disclosure may be better understood by reference to the following description of various examples of the present disclosure taken in conjunction with the accompanying drawings, wherein:

FIG. 1 is a block diagram illustrating an example health and environmental exposure and notification communication network operable to control one or more elements of a health and environmental exposure and notification system.

FIG. 2 is an illustration of an example scenario of a system/technique for providing health and/or environmental exposure tracking and/or notifications.

FIG. 3 is an illustration of an example scenario of a system/technique for providing health and/or environmental exposure tracking and/or notifications.

FIG. 4 is a block diagram of a hardware configuration of an example device that may control one or more parts of any health and environmental exposure and notification system device of FIG. 1.

FIG. 5 is an illustration of an example scenario of a system/technique for providing health and/or environmental exposure tracking and/or notifications.

FIG. 6 is an illustration of an example scenario of a system/technique for providing health and/or environmental exposure tracking and/or notifications.

FIG. 7 is an illustration of an example scenario of a system/technique for providing health and/or environmental exposure tracking and/or notifications.

FIG. 8 is an illustration of an example scenario of a system/technique for providing health and/or environmental exposure tracking and/or notifications.

FIG. 9 is an example flow chart of one example data collection Application flow which can collect Inertial Measurement Unit (IMU) data via Bluetooth.

FIG. 10 is an example flow chart of one example of uploading data from the data collection Application to the cloud.

FIG. 11 is an example flow chart which processes acquired health sensor data (e.g., brushing location data).

FIG. 12 is an illustration of an example process/technique to estimate a user's UV exposure based on the user's location information and the electronic device/smartphone's GPS signal strength.

FIG. 13 is an illustration of an example process/technique to estimate an accumulated UV exposure over time.

FIG. 14 is an example of a daily UV index from smartphone-based weather Applications and/or specialized UV tracking Applications.

FIG. 15A to FIG. 15F are example illustrations of wearable UV-tracking devices.

FIG. 16A to FIG. 16C is an example illustration of electronic device/smartphone indoor/outdoor detection/determination and/or scene detection/determination.

FIG. 17 is an illustration of an example sun exposure presentation via a daily UV index per an electronic device/smartphone weather Application.

FIG. 18 is an illustration of an example illustration of tracking GPS location changes/movements of an electronic device/smartphone to estimate an electronic device user's activity.

### DETAILED DESCRIPTION

For the purposes of promoting an understanding of the principles of the present disclosure, reference may now be made to the examples illustrated in the drawings, and specific language may be used to describe the same. It may nevertheless be understood that no limitation of the scope of this disclosure is thereby intended.

The following description of the preferred embodiment(s) is merely exemplary in nature and is in no way intended to limit the invention or inventions. The description of illustrative embodiments is intended to be read in connection with the accompanying drawings, which are to be considered part of the entire written description. In the description of the exemplary embodiments disclosed herein, any reference to direction or orientation is merely intended for convenience of description and is not intended in any way to limit the scope of the present inventions. Relative terms such as “lower,” “upper,” “horizontal,” “vertical,” “above,” “below,” “up,” “down,” “left,” “right,” “top,” “bottom,” “front” and “rear” as well as derivatives thereof (e.g., “horizontally,” “downwardly,” “upwardly,” etc.) should be construed to refer to the orientation as then described or as shown in the drawing under discussion. These relative terms are for convenience of description only and do not require a particular orientation unless explicitly indicated as such. Terms such as “attached,” “affixed,” “connected,” “coupled,” “interconnected,” “secured” and other similar terms refer to a relationship wherein structures are secured or attached to one another either directly or indirectly through intervening structures, as well as both movable or rigid attachments or relationships, unless expressly described otherwise.

The discussion herein describes and illustrates some possible non-limiting combinations of features that may exist alone or in other combinations of features. Furthermore, as used herein, the term “or” is to be interpreted as a logical operator that results in true whenever one or more of its operands are true. Furthermore, as used herein, the phrase “based on” is to be interpreted as meaning “based at least in part on,” and therefore is not limited to an interpretation of “based entirely on.”

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Features of the present inventions may be implemented in software, hardware, firmware, or combinations thereof. The computer programs described herein are not limited to any particular embodiment, and may be implemented in an operating system, application program, foreground or background processes, driver, or any combination thereof. The computer programs may be executed on a single computer or server processor or multiple computer or server processors.

Processors described herein may be any central processing unit (CPU), microprocessor, micro-controller, computational, or programmable device or circuit configured for executing computer program instructions (e.g., code). Various processors may be embodied in computer and/or server hardware of any suitable type (e.g., desktop, laptop, notebook, tablets, cellular phones, etc.) and may include all the usual ancillary components necessary to form a functional data processing device including without limitation a bus, software and data storage such as volatile and non-volatile memory, input/output devices, graphical user interfaces (GUIs), removable data storage, and wired and/or wireless communication interface devices including Wi-Fi, Bluetooth (e.g., Bluetooth classic, Bluetooth low energy), LAN, etc.

Computer-executable instructions or programs (e.g., software or code) and data described herein may be programmed into and tangibly embodied in a non-transitory computer-readable medium that is accessible to and retrievable by a respective processor as described herein which configures and directs the processor to perform the desired functions and processes by executing the instructions encoded in the medium. A device embodying a programmable processor configured to such non-transitory computer-executable instructions or programs may be referred to as a “programmable device”, or “device”, and multiple programmable devices in mutual communication may be referred to as a “programmable system.” It should be noted that non-transitory “computer-readable medium” as described herein may include, without limitation, any suitable volatile or non-volatile memory including random access memory (RAM) and various types thereof, read-only memory (ROM) and various types thereof, USB flash memory, and magnetic or optical data storage devices (e.g., internal/external hard disks, floppy discs, magnetic tape CD-ROM, DVD-ROM, optical disk, ZIP™ drive, Blu-ray disk, and others), which may be written to and/or read by a processor operably connected to the medium.

In or more scenarios, examples may be embodied in the form of computer-implemented processes and apparatuses such as processor-based data processing and communication systems or computer systems for practicing those processes. The disclosed subject matter may also be embodied in the form of software or computer program code embodied in a non-transitory computer-readable storage medium, which when loaded into and executed by the data processing and communications systems or computer systems, the computer program code segments configure the processor to create specific logic circuits configured for implementing the processes.

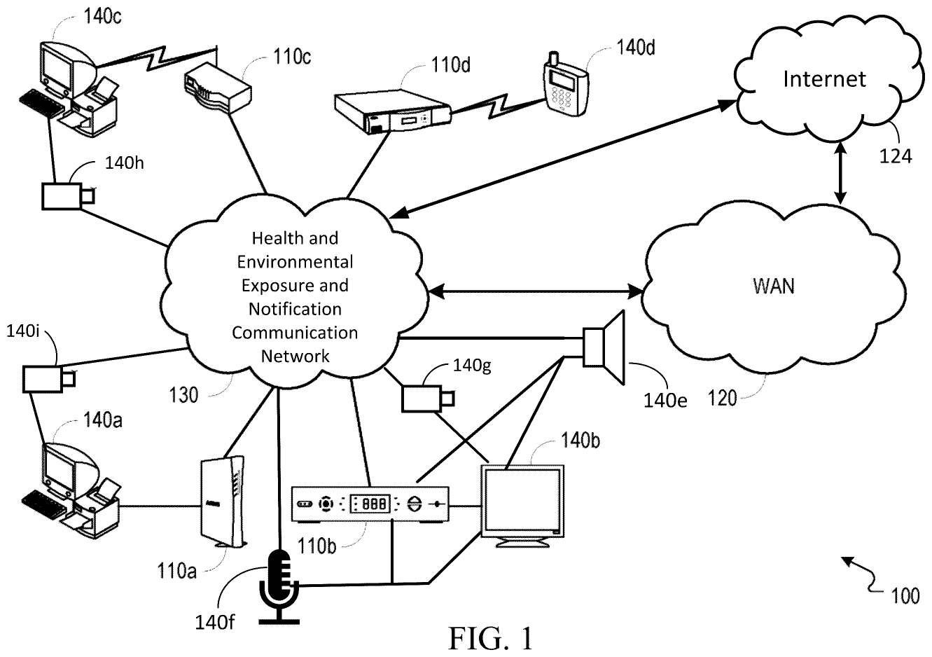

FIG. 1 is a block diagram illustrating an example Health and Environmental Exposure and Notification Communication Network System (HEENCNS) 100 operable to monitor and/or control one or more parts of an Health and Environmental Exposure and Notification System (HEENS). One or more of digital and/or analog control signals, electronic content, various input signals, and or various output signals, among other condition monitoring system information may be communicated from/across/among the Health and Environmental Exposure and Notification Communication Network System (HEENCNS) 100. One or more of discrete control and/or continuous control schemes, techniques, and/or algorithms may be processed/performed by/across/from the Health and Environmental Exposure and Notification Communication Network System (HEENCNS) 100.

Electronic content may include media content, social media content, SMS content, electronic documents, device-to-device communications, streaming media content, digital image still frames, digital streaming video, Internet/cloud-based, edge-based, electronic applications/services/databases, electronic communications/services (e.g., video/audio conferencing), Internet-based electronic services, virtual reality content and/or services, augmented reality content and/or services, media captioning content and/or services, electronic commerce, video components/elements of electronic content, and/or audio components/elements of electronic content, among other types of electronic content.

In one or more scenarios, HEENCNS devices 110a-d transmit/receive signals and/or communications and/or may receive data service(s) from a wide area network (WAN) 120 via a connection to a Health and Environmental Exposure and Notification Communication Network (HEENCN) 130. The one or more nodes of Health and Environmental Exposure and Notification Communication Network (HEENCN) 130 and/or the WAN 120 may communicate with one or more cloud-based nodes (not shown) via the Internet 124.

The Health and Environmental Exposure and Notification Communication Network System (HEENCNS) 100 devices can include, for example, a modem 110a, a process control device/logic controller 110b, a wireless router including an embedded modem 110c, or a media gateway 110d, among many others (e.g., digital subscriber line (DSL) modem, voice over internet protocol (VOIP) terminal adapter, video game console, digital versatile disc (DVD) player, communications device, hotspot device, etc.). The Health and Environmental Exposure and Notification Communication Network (HEENCN) 130, for example, can be a hybrid fiber-coaxial (HFC) network, a local area network (LAN), a wireless local area network (WLAN), a cellular network, and/or a personal area network (PAN), as well as others. As used herein, an Health and Environmental Exposure and Notification Control Device (EENCD) may be any of the devices 110a-110d and/or 140a-140i, an Internet Gateway, a router device, a set-top box (STB), a process control device/logic controller, a smart media device (SMD), a cloud computing device, any type of EENCD, and/or any other suitable device (e.g., wired and/or wireless) that may be configured to perform one or more of the techniques and/or functionality disclosed herein, for example.

The HEENCNS 100 devices can facilitate communications between the WAN 120 and devices 140a-140i. A cable modem or embedded MTA (eMTA) 110 a can facilitate communications between the WAN 120 and a computer 140a. A process control device/logic controller 110b can facilitate communications between the WAN 120 and a television/monitor/display 140b (e.g., a media presentation device, a graphical user interface, a process control interface, etc.) and/or a digital video recorder (DVR). A wireless router 110c can facilitate communications between a computer 140c and the WAN 120.

The media gateway 110d can facilitate communications between a mobile device 140d (e.g., a tablet computing device, a smartphone, a personal digital assistant (PDA) device, a laptop computing device, etc.; one or more devices being PC-based, iOS-based, Linux-based, and/or Android-based, etc.) and the WAN 120. One or more speaker devices (e.g., sound radiation devices/systems) 140e may be in communication with the Health and Environmental Exposure and Notification Communication Network (HEENCN) 130, process control device/logic controller 110b, and/or television/monitor/display 140b, etc. Camera devices 140g, 140h, and/or 140i may be in communication with the computer 140a, the television/display/monitor 140b, the computer 140c, and/or the Health and Environmental Exposure and Notification Communication Network (HEENCN) 130, for example, among other devices and networks.

The one or more speaker devices 140e (e.g., surround sound speakers, home theater speakers, other external wired/wireless speakers, loudspeakers, full-range drivers, subwoofers, woofers, mid-range drivers, tweeters, coaxial drivers, etc.) may broadcast at least an audio component of electronic content/media content, among other audio signals, processes, and/or applications. The one or more speaker devices 140e may possess the capability to radiate sound in pre-configured acoustical/physical patterns (e.g., a cone pattern, a directional pattern, etc.). For example, process control device/logic controller condition monitoring audible alarms may be communicated via one or more of the speaker devices 140e.

One or more microphone devices 140f may be external/standalone microphone devices. The one or more microphone devices 140f may be in communication with the Health and Environmental Exposure and Notification Communication Network (HEENCN) 130, process control device/logic controller 110b, television/display/monitor 140b, computer 140a, computer 140c, mobile device 140a, etc. Any of the devices 110a-110d and/or devices 140a-140i may include internal microphone devices. The one or more speaker devices 140e (e.g., “speakers”) and/or the one or more microphone devices 140f (e.g., “microphones”, that may be “high quality” devices such as far field microphones, noise-cancelling microphones, shotgun microphones, dynamic microphones, ribbon microphones, and/or various size diaphragm microphones, Bluetooth™-based remote/control devices, RF4CE-based remote/control devices, etc.) may have wired and/or wireless connections (e.g., Bluetooth, Wi-Fi, private protocol communication network, etc.) to any of the other devices 140a-140i, the Health and Environmental Exposure and Notification Communication Network (HEENCN) 130, the WAN 120, and/or the Internet 124.

The camera devices 140g-140i may provide digital video input/output capability for one or more of the devices 110a-110d and/or devices 140a-140d. The camera devices 140g-140i may communicate with any of the devices 110a-110d and/or devices 140a-140f, perhaps for example via a wired and/or wireless connection. One or more of the camera devices 140g-140i may capture digital images, digital video streams, and/or may scan images of various kinds, such as Universal Product Code (UPC) codes and/or Quick Response (QR) codes, for example, among other images. One or more of the camera devices 140g-140i may provide for video input/output for video monitoring (e.g., may serve as webcams or the like), for example, among other video functions.

Any of the camera devices 140g-140i may include microphone devices and/or speaker devices. The input/output of any of the camera devices 140g-140i may include audio signals/packets/components, perhaps for example separate/separable from, or in some (e.g., separable) combination with, the video signals/packets/components of any of the camera devices 140g-140i.

One or more of the camera devices 140g-140i may detect the presence of one or more subjects and/or things (e.g., food containers) that may be proximate to the camera devices 140g-140i and/or that may be in the same general space (e.g., the same room, same space, same room, same delimited area, etc.) as the camera devices 140g-140i. One or more of the camera devices 140g-140i may gauge a general activity level (e.g., high activity, medium activity, and/or low activity) of one or more subjects that may be detected by the camera devices 140g-140i. One or more of the camera devices 140g-140i may detect one or more general characteristics (e.g., height, body shape, skin color, pulse, heart rate, breathing count, object size, object volume, object bulk, etc.) of the one or more subjects and/or things detected by the camera devices 140g-140i. One or more of the camera devices 140g-140i may be configured to recognize one or more specific subjects and/or objects, for example. One or more of the camera devices 140g-140i may be configured to detect a subject's attention/gaze toward another subject (e.g., detecting a subject and/or object that may correspond to a subject's attention/gaze toward another subject or object).

One or more of the camera devices 140g-140i may be use wireless communication with any of the devices 110a-110d and/or 140a-140d, such as for example Bluetooth™ and/or Wi-Fi™ among other wireless communication protocols. One or more of the camera devices 140g-140i may be external to any of the devices 110a-110d and/or devices 140a-140d. One or more of the camera devices 140g-140i may be internal to any of the devices 110a-110d and/or devices 140a-140d.

One or more of the camera devices 140g-140i may be an industrial vision camera device. The vision camera may be a gigabit Ethernet compatible device (e.g., 10 GB Ethernet, or the like). The vision camera may function in black & white and/or color. The vision camera may have a capacity of at least 8.8 megapixel, or the like. The vision camera may have a resolution of 4096×2160 pixel, or the like. For example, the vision camera may be a (e.g., manufactured by Baumer such as a VLXT-90C.I LX series, or like/equivalent or other device as mentioned herein) may capture product images in various forms such as digital still image frames and/or video streams, etc., perhaps for example from zero to ninety-five (95) frames per second (fps). The vision camera may have one or more parameters configurable remotely and/or locally. Camera devices 140g-140i may include color/RGB, black & white/grayscale, infrared, and/or devices capable of measuring non-visible light spectrums, such as multi-spectral and/or hyper-spectral and/or Depth/3D cameras.

Health and Environmental Exposure and Notification Communication Network System (HEENCNS) 100 devices such as process control device/logic controller devices, media gateway devices, among others, may support visual and/or voice interface with users, viewers, and/or Health and Environmental Exposure and Notification Communication Network (HEENCN) 130 operators. This interface may support smart enhancement to the user/viewer/operator experience, for example in the subject nutrition dispensing monitoring network environment, or in any network environment. One or more traditional and/or current viewer experiences can be enriched to utilize visual and/or voice interface, perhaps for example to derive smart actions and/or results.

In one or more scenarios, any of the devices 110a-110d, 140a-140i, among other devices, may be used to implement any of the capabilities, techniques, methods, and/or devices described herein.

The WAN network 120 and/or the Health and Environmental Exposure and Notification Communication Network (HEENCN) 130 may be implemented as any type of wired and/or wireless network, including a local area network (LAN), a wide area network (WAN), a global network (the Internet), etc. Accordingly, the WAN network 120 and/or the Health and Environmental Exposure and Notification Communication Network (HEENCN) 130 may include one or more communicatively coupled network computing devices (not shown) for facilitating the flow and/or processing of network communication traffic via a series of wired and/or wireless interconnections. Such network computing devices may include, but are not limited, to one or more access points, routers, switches, servers, computing devices, and/or storage devices, etc.

Without the capabilities, techniques, methods, systems, and/or devices described herein, the skilled artisan would not appreciate how to apprise a user of an electronic device/smartphone to determine their UV exposure without a specific UV exposure sensor, or the like. Furthermore, the skilled artisan would not appreciate how to provide a user with health and/or environmental exposure information by way of the user's preferred social media application(s) as accessed/initiated by the user at the user's discretion.

Connected health devices with sensors, such as smart toothbrushes, UV sensors, health and fitness trackers, toothbrushes, etc., may provide a wealth of information to end users to monitor their health, their environment, and/or physical activities. These devices may have a companion Application to be installed and used on a smart mobile/electronic device, such as a smartphone or digital tablet. The specific Application serves as an interface between the connected device and end users to provide data and feedback to the end users. However, with the increasing number of Applications on users' smartphones, the additional requirements of memory, data storage, and power consumption become an issue. Moreover, many end users become unwilling to install additional Applications on their devices specifically for connected devices. Even when users/end users do install the Applications on their devices, they rarely or simply do not open or engage with the Application to get the benefits of data/information collected by the connected devices, as well as the recommendations/feedback from the Application. Therefore, a different approach to collect the user data, process the data, then provide recommendations and feedback to users without users having to open and engage with the device specific Applications may be useful.

Described herein are architectures for connected devices (health and wellness trackers, IoT and smart devices, etc.) that, on a routine daily basis, users may interact with these connected devices through a social media Application of users' choice. The social media Application could be one of those popular Applications such as FaceBook, Instagram, Twitter, TikTok, Snapchat, LinkedIn, WhatsApp, WeChat, Pinterest, Reddit, Telegram etc. Since users normally engage with the social media Applications of their choice voluntarily and/or multiple times a day, the user feedback and/or recommendations may naturally reach users without additional efforts from the users' side (e.g., open the specific Applications for the connected devices). It also makes real time feedback possible if the users choose to allow pop notifications from those specific social media Applications on their smartphones, for example.

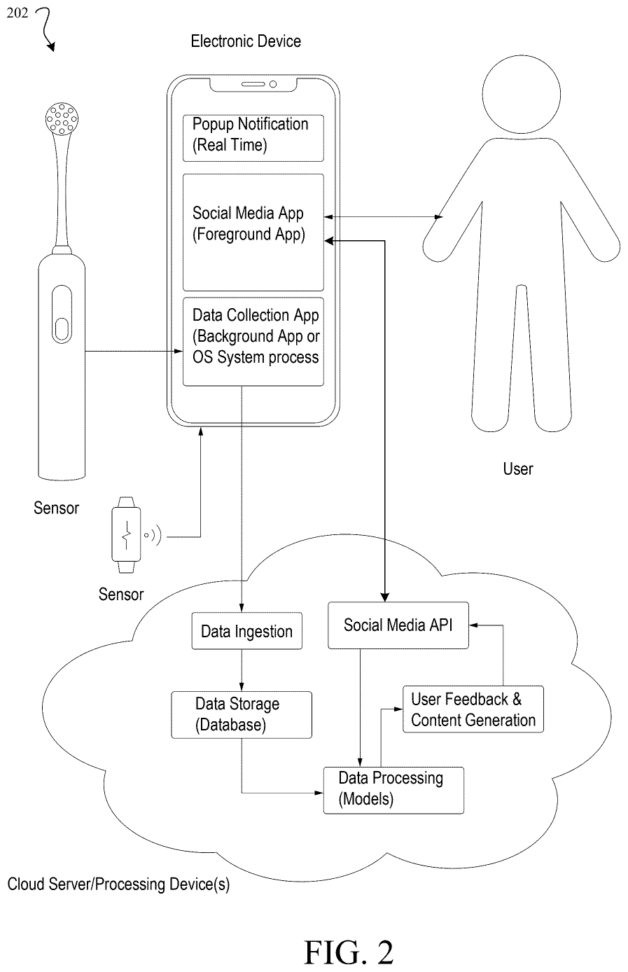

FIG. 2 is an illustration of an example scenario 202 of a system/technique for providing health and/or environmental exposure tracking and/or notifications. During the setup process on the electronic device/smartphone for the connected device, users install a light weight data collection Application that may run in the background and/or it may be a customized system process of the operating system (e.g., also running in the background). This data collection Application may (e.g., may always) run in the background and may communicate with the one or more connected devices (e.g., sensor, data services providers, other processing devices, etc.) through one or multiple wireless protocols (e.g., such as Bluetooth, BLE, Wifi). The data collection Application has a relatively small (e.g., very small) footprint (minimum memory/storage requirements) and/or low energy consumption, for example since it may (e.g., may only) perform data collection, perhaps without relatively heavy data processing and/or user interface components/functions.

A data collection Application may connect to a cloud server and/or upload the collected data from connected devices to a Data Ingestion module (e.g., in a Cloud Server/Processing Device) through standard wireless protocols (e.g., Wifi, cellular network, Matter, Amazon Sidewalk). The raw data may be processed and/or stored into a Database on the cloud server. The newly acquired data and/or historical data may be utilized by the mathematical models in the data processing engine to create user desirable insight. This information and/or insight may be passed to the User Feedback & Content Generation engine to create text, audio, images and videos for the users to consume. This engine may be an adaptive generation engine that may determine the content, format, and/or frequency of user feedback, based on the data collected by the connected devices and/or the specific user's personal interactions with previous content.

The Social Media API may maintain two-way communication with user's social media Applications to transmit contents created based on connected devices (e.g., text, audio, image and/or video) and/or may collect users' feedback, which may be fed into The Data Processing engine again to create real time responses through the previously described mechanism.

In one or more scenarios, users may accept the “friend request” from the social media accounts of connected device manufacturers. This can be accomplished by using a QR code and/or hyperlink and/or a normal weblink so the users can connect with the social media account of that particular connected device(s). This also creates the opportunity for users to share their experiences on this connected device with their friends and/or family members if they are satisfied with the product and/or service to create a user network group that they can share their experiences and compete to see who has the best performance and/or nudge their friends and family members to maintain good habits and healthy lifestyle.

Data Collection Applications are described herein. Different connected health devices may have different data collection Applications. They may have similar flows such as device setup, establish connections, data transfer, monitor connection, and/or handle disconnection, etc. Device setup may be more (e.g., tightly) related with the specific device. The connections might be over the Bluetooth, WiFi network, etc.

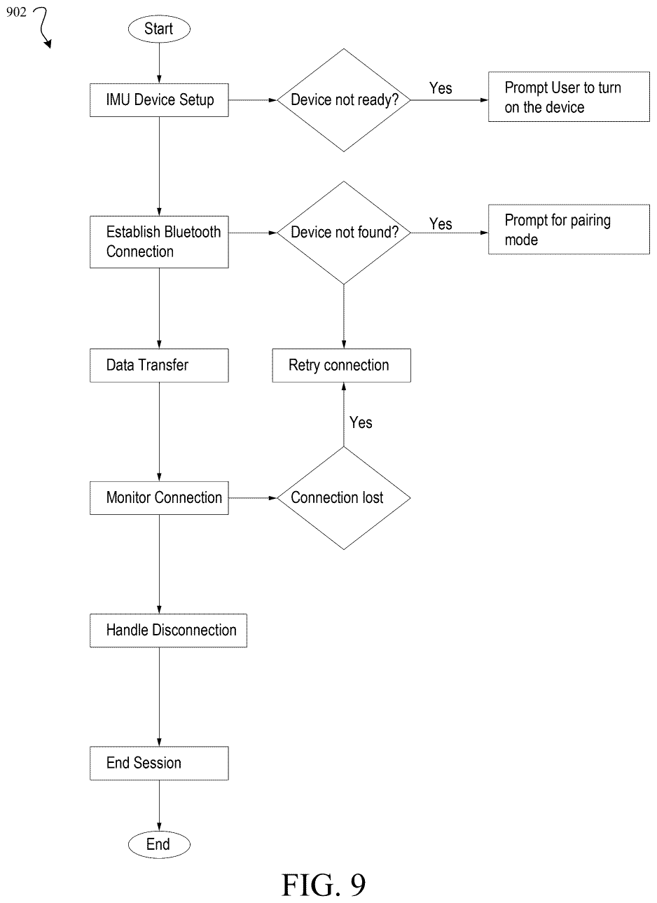

FIG. 9 shows the flow chart 902 of one example data collection Application which can collect Inertial Measurement Unit (IMU) data via Bluetooth, for example. IMU device setup and establishment of a Bluetooth connection are described herein. The following are some pseudo code for IMU device setup and establishment of a Bluetooth connection on Android and iOS:

```
  Android (Kotlin):
  // Initialize BluetoothAdapter
  val bluetoothManager = getSystemService(Context.BLUETOOTH_SERVICE) as
BluetoothManager
  val bluetoothAdapter: BluetoothAdapter? = bluetoothManager.adapter
  // Start scanning for devices
  bluetoothAdapter?.startLeScan { device, rssi, scanRecord −>
    // Check if the found device is the IMU and connect
  }
  // Connect to the device and set up communication
  // ...
  iOS (Swift)
  import CoreBluetooth
  class BluetoothManager: NSObject, CBCentralManagerDelegate {
    var centralManager: CBCentralManager!
    override init( ) {
      super.init( )
      centralManager = CBCentralManager(delegate: self, queue: nil)
    }
    func centralManagerDidUpdateState(_ central: CBCentralManager) {
      if central.state == .poweredOn {
        // Start scanning for devices
        central.scanForPeripherals(withServices: nil, options: nil)
      }
    }
    func centralManager(_ central: CBCentralManager, didDiscover peripheral:
CBPeripheral, advertisementData: [String: Any], rssi RSSI: NSNumber) {
      // Check if the found peripheral is the IMU and connect
    }
    // Connect to the peripheral and set up communication
    // ...
  }
```

Data transfer is described herein. Once the connection between the device and the data collection Application is established, the following are some code snippets that demonstrate the useful steps of transferring the data from a device to the data collection Application.

```
  BluetoothAdapter bluetoothAdapter = BluetoothAdapter.getDefaultAdapter( );
  if (bluetoothAdapter == null) {
    // Device doesn't support Bluetooth
  }
  // Enable Bluetooth if it's not enabled
  if (!bluetooth Adapter.isEnabled( )) {
    Intent enableBtIntent = new
Intent(BluetoothAdapter.ACTION_REQUEST_ENABLE);
    startActivityForResult(enableBtIntent, REQUEST_ENABLE_BT);
  }
  // Assuming you've already paired the IMU device, get it by its name or address
  BluetoothDevice imuDevice = null;
  Set<BluetoothDevice> pairedDevices = bluetoothAdapter.getBondedDevices( );
  if (pairedDevices.size( ) > 0) {
    for (BluetoothDevice device : pairedDevices) {
      if (device.getName( ).equals(“IMU_Device_Name”) ||
device.getAddress( ).equals(“IMU_Device_Address”)) {
        imuDevice = device;
        break;
      }
    }
  }
  Create a BluetoothSocket and connect to the IMU device:
  // UUID that corresponds to the IMU device's SPP (Serial Port Profile) service
  UUID imuUUID = UUID.fromString(“00001101-0000-1000-8000-00805f9b34fb”);
  BluetoothSocket bluetoothSocket = null;
  try {
    bluetooth Socket = imuDevice.createRfcommSocketToServiceRecord(imuUUID);
    bluetoothSocket.connect( );
  } catch (IOException e) {
    // Handle connection failure
  }
  Transfer data from the IMU device:
  // Create a separate thread to manage the connection and data transfer
  private class ConnectedThread extends Thread {
    private final InputStream mmInStream;
    private final OutputStream mmOutStream;
    public ConnectedThread(BluetoothSocket socket) {
      InputStream tmpIn = null;
      OutputStream tmpOut = null;
      try {
        tmpIn = socket.getInputStream( );
        tmpOut = socket.getOutputStream( );
      } catch (IOException e) {
        // Handle exception
      }
      mmInStream = tmpIn;
      mmOutStream = tmpOut;
    }
    public void run( ) {
      byte[ ] buffer = new byte; [1024]// Buffer to store the stream
      int bytes; // Bytes returned from read( )
      // Keep listening to the InputStream until an exception occurs
      while (true) {
        try {
          // Read from the InputStream
          bytes = mmInStream.read(buffer);
          // Send the obtained bytes to the UI activity
          // You can use a Handler or other means to process the data
        } catch (IOException e) {
          // Handle disconnection
          break;
        }
      }
    }
    // Call this method from the main activity to send data to the remote device
    public void write(byte[ ] bytes) {
      try {
        mmOutStream.write(bytes);
      } catch (IOException e) {
        // Handle exception
      }
    }
    // Call this method from the main activity to shut down the connection
    public void cancel( ) {
      try {
        bluetoothSocket.close( );
      } catch (IOException e) {
        // Handle exception
      }
    }
  }
```

Start the thread to manage the connection and data transfer:

ConnectedThread connectedThread=new ConnectedThread (bluetoothSocket);

connectedThread.start( )

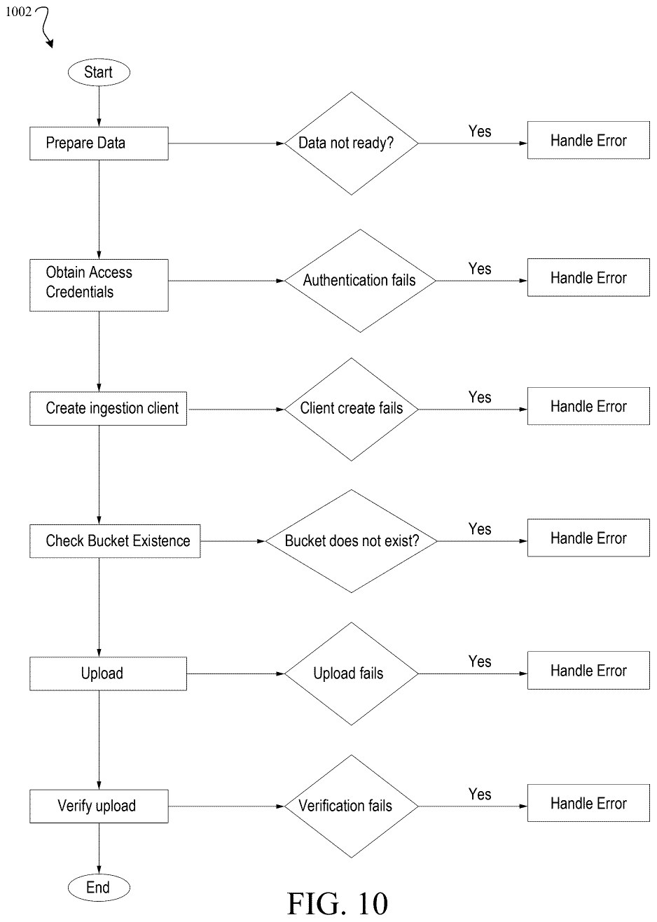

Data Ingestion and Data Storage is described herein. Uploading data from the data collection Application to the cloud involves several steps, including choosing a cloud storage provider, integrating their API or SDK into the Application, and implementing the upload functionality. FIG. 10 shows the general outline 1002 of the process.

### Example Code Snippets

```
  Uploading a file to AWS S3 using the AWS SDK for Java (Android):
  AmazonS3 s3Client = new AmazonS3Client(new
BasicAWSCredentials(“ACCESS_KEY_ID”, “SECRET_ACCESS_KEY”));
  File fileToUpload = new File(“/path/to/your/file.txt”);
  s3 Client.putObject(new PutObjectRequest(“your-bucket-name”, “file.txt”,
fileToUpload));
  Uploading a file to Firebase Cloud Storage using the Firebase SDK for iOS (Swift):
  let storage = Storage.storage( )
  let storageRef = storage.reference( )
  let fileRef = storageRef.child(“path/to/your/file.txt”)
  let localFile = URL(fileURLWithPath: “/path/to/your/file.txt”)
  fileRef.putFile(from: localFile, metadata: nil) { metadata, error in
    guard let metadata = metadata else {
      // Handle error
      return
    }
    // File uploaded successfully
  }
```

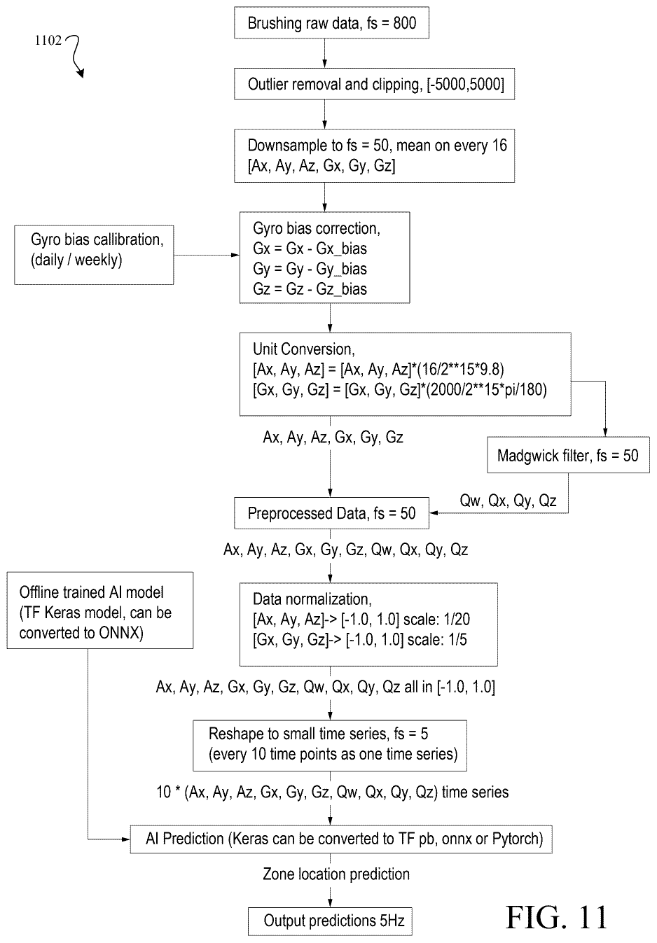

Data Processing is described herein. The data processing flow might be different depending on the data types. FIG. 11 shows an example flow chart 1102 which processes brushing location health care data. After the data is processed, the information can be obtained such as how long the user has been brushed on each zone, upper jaw or lower jaw, left or right, occlusal surface, buccal side, and/or lingual side etc. The user may be reminded that some zones might be missed, brushed too short time, etc.

```
  Code snippets for outlier removal and clipping:
  def clip(df, min_value =−5000, max_value=5000):
    columns_to_convert = [‘Ax’, ‘Ay’, ‘Az’, ‘Gx’, ‘Gy’, ‘Gz’]
    df[columns_to_convert] = df[columns_to_convert].clip(lower=min_value,
upper=max_value)
    return df
  Code snippet for downsample:
  def downSampleByMean(df, with_label=True, downsample_step_size=16):
    # Add a new column for grouping every downsample_step_size rows
    df[‘Group’] = (np.arange(len(df)) // downsample_step_size) + 1
    # Define custom aggregation functions
    if with_label:
      agg_funcs = {
        ‘Ax’: ‘mean’,
        ‘Ay’: ‘mean’,
        ‘Az’: ‘mean’,
        ‘Gx’: ‘mean’,
        ‘Gy’: ‘mean’,
        ‘Gz’: ‘mean’,
        ‘Label’: lambda x: x.iloc[0] # Pick the first value in the group
      }
    else:
      agg_funcs = {
        ‘Ax’: ‘mean’,
        ‘Ay’: ‘mean’,
        ‘Az’: ‘mean’,
        ‘Gx’: ‘mean’,
        ‘Gy’: ‘mean’,
        ‘Gz’: ‘mean’
      }
    # Group by ‘Group’ and aggregate using custom functions
    aggregated_df = df.groupby(‘Group’). agg(agg_funcs)
    # print(“Original DataFrame:”)
    # print(df.head(40)) # Displaying first 20 rows for demonstration
    # print(“\nDownsampled DataFrame:”)
    # print(aggregated_df.head(3))
    # print(“down sample size: { }”.format(aggregated_df.shape[0]))
    # print(“total time in seconds: { }”.format(aggregated_df.shape[0] *
downsample_step_size / 800.0))
    return aggregated_df
  Code snippets for Gyro correction and unit conversion:
  defbiasAndUnitConversion(raw_ax, raw_ay, raw_az, raw_gx, raw_gy, raw_gz, bias_gx,
bias gy, bias_gz):
    “““.vscode/
    Bias and unit conversion for the IMU data
      - +/− 16g sensitivity for the accelerometer, 2\hat{ }15 is the bit width of the ADC value
      - 9.8 is the gravity in m/s2
      - +/− 2000 is the range of gyro deg/sec, 2\hat{ }15 is the bit width of the ADC value
    ”””
    scale_factor_a = 16 / 2**15 * 9.8 # full scale range of 16g / max 16-bit value/2 (+/−)
    ax = raw_ax * scale_factor_a
    ay = raw_ay * scale_factor_a
    az = raw_az * scale_factor_a
    scale_factor_g = 2000 / 2**15 * np.pi / 180
    gx = (raw_gx − bias_gx) * scale_factor_g
    gy = (raw_gy − bias_gy) * scale_factor_g
    gz = (raw_gz − bias_gz) * scale_factor_g
    return ax, ay, az, gx, gy, gz
  Code snippets for data normalization:
  def dataNormalization(df, max_accl=20.0, max_gyro=5.0):
    clip_ranges = {‘Ax’: (−max_accl, max_accl), ‘Ay’: (−max_accl, max_accl), ‘Az’: (−
max_accl, max_accl),
            ‘Gx’: (−max_gyro, max_gyro), ‘Gy’: (−max_gyro, max_gyro), ‘Gz’: (−
max_gyro, max_gyro) }
    # Clip the columns to their respective ranges
    for column, (min_val, max_val) in clip_ranges.items( ):
      df[column]= df[column].clip(min_val, max_val)
    df[‘Ax’] = df[‘Ax’] / max_accl
    df[‘Ay’] = df[‘Ay’] / max_accl
    df[‘Az’] = df[‘Az’] / max_accl
    df[‘Gx’] = df[‘Gx’] / max_gyro
    df[‘Gy’] = df[‘Gy’] / max_gyro
    df[‘Gz’] = df[‘Gz’] / max_gyro
    return df
  Code snippets for reshaping to small time series:
  def grouping(X, y, group_size, X_attr_size=10, with_label=True):
    “““
    Grouping: Group multiple data points as one time series
    - After grouping, the time series can be flattened and feed into a Dense layer
      - For Dense: after grouping, input_shape=(n_timesteps * n_features, )
    - After grouping, the time series can be directly feed into a LSTM layer.
      - For LSTM: input_shape=(n_timesteps,n_features)
    :param X: data
    :param y: label
    :group_size: the number of X to be grouped together, same applied to y
    :X_attr_size: attribute size of X
    :return: Grouped X and y
    ”””
    X_grouped = np.array([ ])
    y_grouped = np.array([ ])
    row, _= X.shape
    i=0
    while i < row and group_size + i < row:
      if with label:
        y_group = y[i:group_size+i]
      X_group = np.reshape(X[i:group_size+i, :], (1, X_attr_size*group_size))
      # Make sure the same group has the same label
      if with_label:
        same_value = np.all(y_group == y_group[0])
      else:
        same_value = Tru
      if same_value and y_group[0] != −1:
        if X_grouped.size == 0:
          X_grouped = X_group
        else:
          X_grouped = np.append(X_grouped, X_group, axis=0)
        if with label:
          if y_grouped.size == 0:
              y_grouped = y_group[0]
          else:
              y_grouped = np.append(y_grouped, y_group[0])
      i = i + group_size
    return X_grouped, _groped
  Code snippets for AI prediction.
  def predict(model_filepath, X_val, y_val, class_num):
    “““
    Load a trained model and make predictions on dataset [X_val, y_val]
    ”””
    # Load the best model
    best_model = tf.keras.models.load_model(model_filepath)
    # Get predictions on validation set using best_model
    val_preds = best_model.predict(X_val)
    # Get most likely class index
    val_preds = np.argmax(val_preds, axis=1)
    # Calculate confusion matrix
    cm = confusion_matrix(y_val, val_preds)
    Plot confusion matrix
    df_cm = pd.DataFrame(cm, index=range(class_num), columns=range(class_num))
    plt.figure(figsize=(10,8))
    sns.heatmap(df_cm, annot=True, fmt=“d”, cmap=“Blues”)
    plt.xlabel(“predicted”)
    plt.ylabel(“True”)
    plt.title(“Confusion Matrix”)
    plt.show( )
    # Calculate and print accuracy
    accuracy_to_print — np.trace(cm) / float(np.sum(cm))
    print(‘Accuracy validation set: {:.4f)}’.format(accuracy_to_print))
    #Print precision and recall per class
    print(‘Recall per class: ’, cm.diagonal( )/cm.sum(axis=1))
    # Print class-wise accuracy
    print(‘Class-wise accuracy: ’, cm.diagonal( )/cm.sum(axis=0))
```

Content Generation is described herein. Once the data is processed, the social media Application can generate content and share the generated content to the user via a message or a popup notification. Below is an example of how a social media create a popup notification:

```
  import android.Application.NotificationChannel;
  import android.Application.NotificationManager;
  import android.content.Context;
  import android.os.Build;
  import androidx.core.Application.NotificationCompat;
  import androidx.core.Application.NotificationManagerCompat;
  public class NotificationHelper {
    public static void displayNotification(Context context, String title, String content) {
      String CHANNEL_ID = “social_media_notifications”;
      String CHANNEL_NAME = “Social Media Application Notifications”;
      NotificationManagerCompat notificationManager =
NotificationManagerCompat.from(context);
      // Create a notification channel for Android Oreo and above
      if (Build.VERSION.SDK_INT >= Build.VERSION_CODES.O) {
        NotificationChannel channel = new NotificationChannel(
            CHANNEL_ID,
            CHANNEL_NAME,
            NotificationManager.IMPORTANCE_DEFAULT
        );
        notificationManager.createNotificationChannel(channel);
      }
      // Create the notification
      NotificationCompat.Builder builder = new NotificationCompat.Builder(context,
CHANNEL_ID)
          .setSmallIcon(R.drawable.ic_notification) // Replace with your notification
icon
          .setContentTitle(title)
          .setContentText(content)
          .setPriority(NotificationCompat.PRIORITY_DEFAULT)
          .setAutoCancel(true); // Dismisses the notification when tapped
      // Show the notification
      int notificationId = 1; // You can use a more complex logic for unique IDs
      notificationManager.notify(notificationId, builder.build( ));
    }
  }
  NotificationHelper.displayNotification(context, “New Message”, “You have a new
message from Colgate!”);
  The following are some example Notification Payloads:
  APNS Payload Example (JSON):
  {
    “aps”: {
      “alert”: {
        “title”: “New Message”,
        “body”: “You missed your upper right inside zone.”
      },
      “badge”: 1,
      “sound”: “default”
    },
    “customData”: {
      “conversationId”: “12345”
    }
  }
  FCM Payload Example (JSON):
  {
    “to”: “device_token”,
    “notification”: {
      “title”: “New Message”,
      “body”: “You missed your upper right inside zone.”
    },
    “data”: {
      “conversationId”: “12345”
    }
  }
```

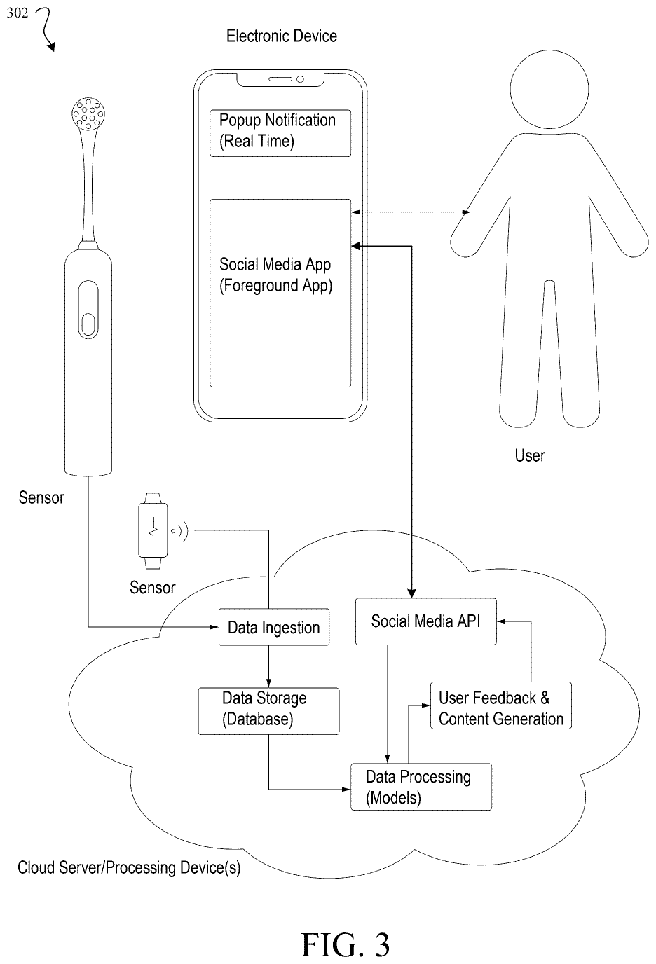

FIG. 3 is an illustration of an example scenario 302 of a system/technique for providing health and/or environmental exposure tracking and/or notifications. In FIG. 3 the connected devices/sensors may interface directly with Cloud Server's Data Ingestion module through standard wireless protocols (e.g., Wifi, cellular network, Matter, Amazon Sidewalk). This configuration may require the connected devices to be equipped with wireless communication components that are capable of direct communication to the Internet. During the initial user setup process, users may “friend” the social media accounts of connected device manufacturers. Then the user may transmit the connected device's unique device ID or serial code to the device manufacturer's social media account, either through a QR code or text/number combination engraved on the connected device itself or on the packaging material or a printout included with the product package. This process may connect the specific social media account to a specific hardware connected device.

The rest of the data flow may be exactly/substantially the same as that described with respect to FIG. 2. There may be an exception in that there may be no need/use to have a background Application and/or process running on the users' smartphones. Users may interact with the social media platform of their choice from the setup to the daily engagement with the connected devices/sensors. This may further reduce the requirements on the user's side and/or there may be no/much resource utilization (e.g., memory, CPU cycle, and/or battery consumption, etc.) on users' smartphones.

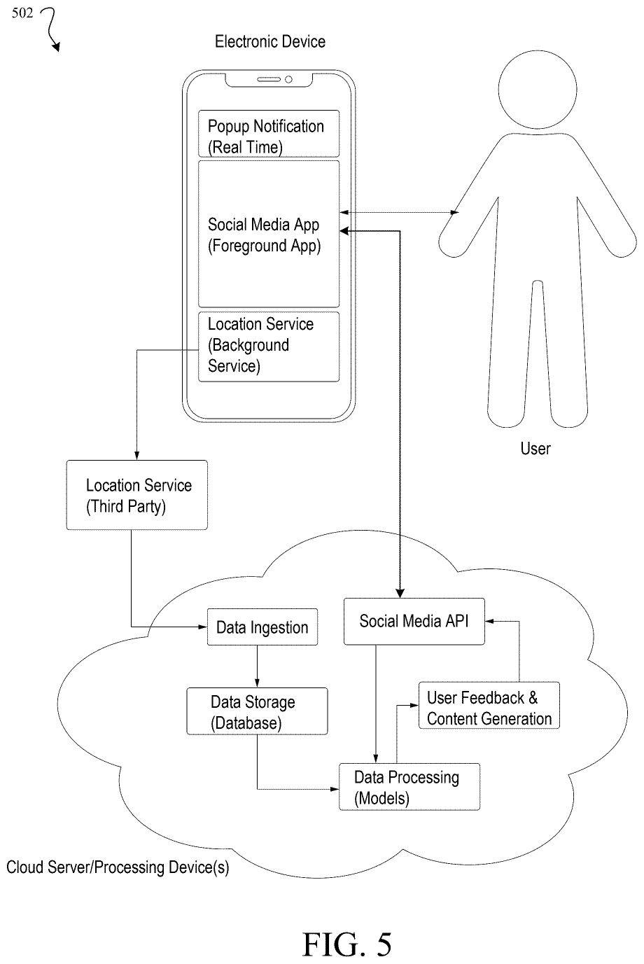

FIG. 5 is an illustration of an example scenario 502 of a system/technique for providing health and/or environmental exposure tracking and/or notifications. In FIG. 5 may be specific for UV exposure estimation and/or reminder of application of sunscreen products, no additional hardware may be required. As illustrated in FIG. 5, during the initial user setup process, users may “friend” the social media accounts of this UV exposure/Sunscreen/Environmental Indicator service provider. The users may enable “Location tracking” on their smartphone, e.g., though Google Map and/or Apple Map. They may give permission for “location sharing” to the social media account of the service provider.

The Data Ingestion module of the service provider's cloud server may fetch the location of the user periodically, may compute its UV exposure based on the specific location (indoor or outdoor, does it have shade, etc.), and/or the UV index from a weather service's API. The UV exposure value may be stored in the Database. The user's location information may be discarded (e.g., immediately) to protect user's privacy. The mathematical models in the Data Processing engine may integrate the users' UV exposure data to determine if a critical threshold may have been reached or exceeded to trigger a message for the user. The User Feedback and Content Generation engine may create text, audio, image, and/or video content to be sent to the user's chosen/selected social media account via Social Media API. Users may receive real time notifications on their electronic device/smartphone via popup and/or directly through their social media Application.

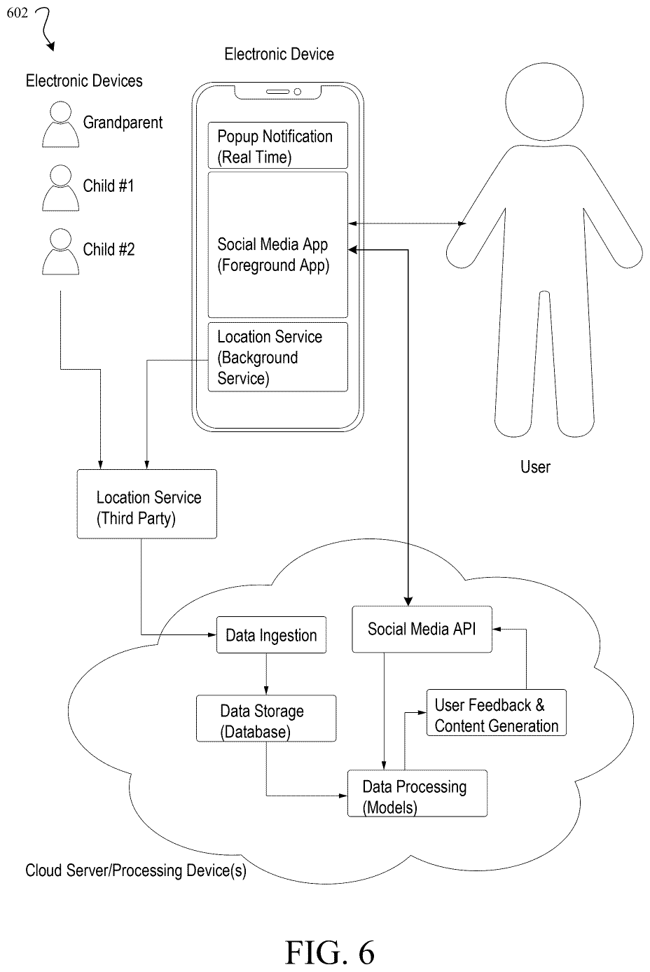

Caregivers (e.g., parents of young children or caregivers of seniors) may choose to provide location trackers (e.g., Apple AirTags) to their care receivers and/or may share their location information to the UV exposure/sunscreen service provider when they create a family profile. FIG. 6 is an illustration of an example scenario 602 of a system/technique for providing health and/or environmental exposure tracking and/or notifications. As illustrated in FIG. 6, the location information of care receivers may be fed into the service provider's Data Ingestion module, and the rest of the data processing and flow may be substantially similar to that as described with respect to FIG. 5. A specific message for a care receiver may be transmitted to the user's social media account if that person's UV exposure may have exceeded the limit and/or that person requires a re-application of sunscreen, among other messages, for example.

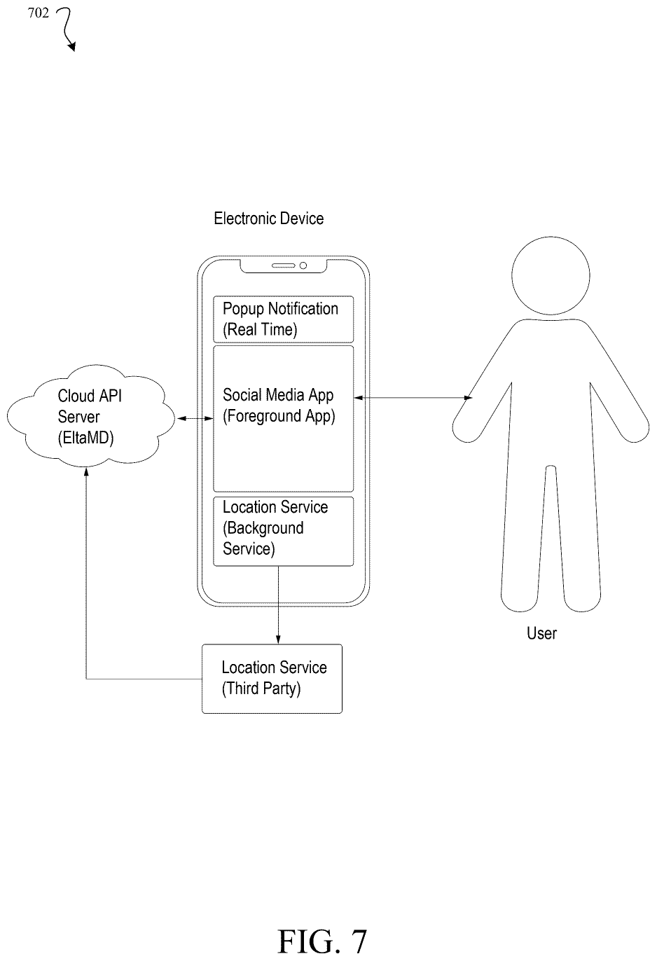

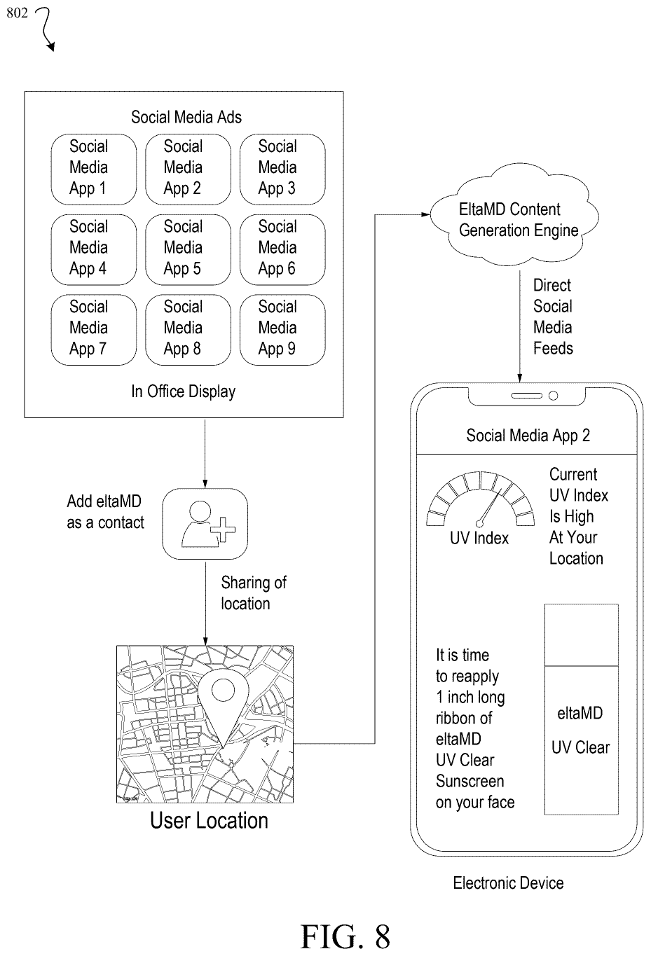

FIG. 7 is an illustration of an example scenario 702 of a system/technique for providing health and/or environmental exposure tracking and/or notifications. For example, one user may subscribe to EltaMD's official (e.g., corporate) account, for example, among other health care advise service providers, and/or may allow sharing location information to EltaMD. EltaMD's server can generate personalized content based on the user's location. FIG. 8 is an illustration of an example scenario 802 of a system/technique for providing health and/or environmental exposure tracking and/or notifications.

Pseudo code to fetch location data from google location service follows:

```
  import android.Manifest;
  import android.content.pm.PackageManager;
  import android.location.Location;
  import android.os.Bundle;
  import androidx.annotation.NonNull;
  import androidx.core.Application.ActivityCompat;
  import androidx.appcompat.Application.AppCompatActivity;
  import com.google.android.gms.location.FusedLocationProviderClient;
  import com.google.android.gms.location.LocationServices;
  import com.google.android.gms.tasks.OnSuccessListener;
  public class MainActivity extends AppCompatActivity {
    private FusedLocationProviderClient fusedLocationClient;
    @Override
    protected void onCreate(Bundle savedInstanceState) {
      super.onCreate(savedInstanceState);
      setContentView(R.layout.activity_main);
      fusedLocationClient = LocationServices.getFusedLocationProviderClient(this);
      // Check for location permissions
      if (ActivityCompat.checkSelfPermission(this,
Manifest.permission.ACCESS_FINE_LOCATION) !=
PackageManager.PERMISSION_GRANTED
          && ActivityCompat.checkSelfPermission(this,
Manifest.permission.ACCESS_COARSE_LOCATION) !=
PackageManager.PERMISSION_GRANTED) {
        // Request permissions if not already granted
        ActivityCompat.requestPermissions(this, new
String[ ]{Manifest.permission.ACCESS_FINE_LOCATION,
Manifest.permission.ACCESS_COARSE_LOCATION}, 1000);
      } else {
        // Permissions are already granted, fetch the location
        fetchLocation( );
      }
    }
    private void fetchLocation( ) {
      fusedLocationClient.getLastLocation( )
        .addOnSuccessListener(this, new OnSuccessListener<Location>( ) {
          @Override
          public void onSuccess(Location location) {
            // Got last known location. In some rare situations this can be null.
            if (location != null) {
              // Logic to handle location object
              double latitude = location.getLatitude( );
              double longitude = location.getLongitude( );
              // Use the latitude and longitude as needed
            }
          }
        });
    }
    @Override
    public void onRequestPermissionsResult(int requestCode, @NonNull String[ ]
permissions, @NonNull int[ ] grantResults) {
      super.onRequestPermissionsResult(requestCode, permissions, grantResults);
      if (requestCode == 1000) {
        if (grantResults.length > 0 && grantResults[0] =
PackageManager.PERMISSION_GRANTED) {
          // Permissions granted, fetch the location
          fetchLocation( );
        } else {
          // Permissions denied, handle the error
        }
      }
    }
  }
```

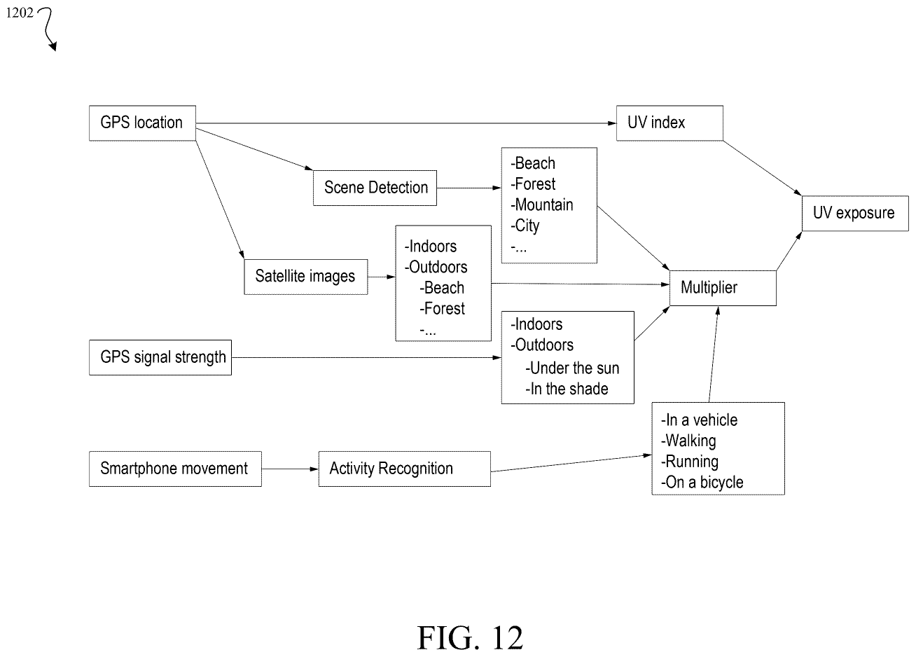

FIG. 12 is an illustration of an example process/technique 1202 to estimate a user's UV exposure based on the user's location information and the electronic device/smartphone's GPS signal strength. FIG. 12 shows how to (e.g., more accurately) estimate a user's UV exposure based on the user's location information and/or the electronic device/smartphone's GPS signal strength, etc.

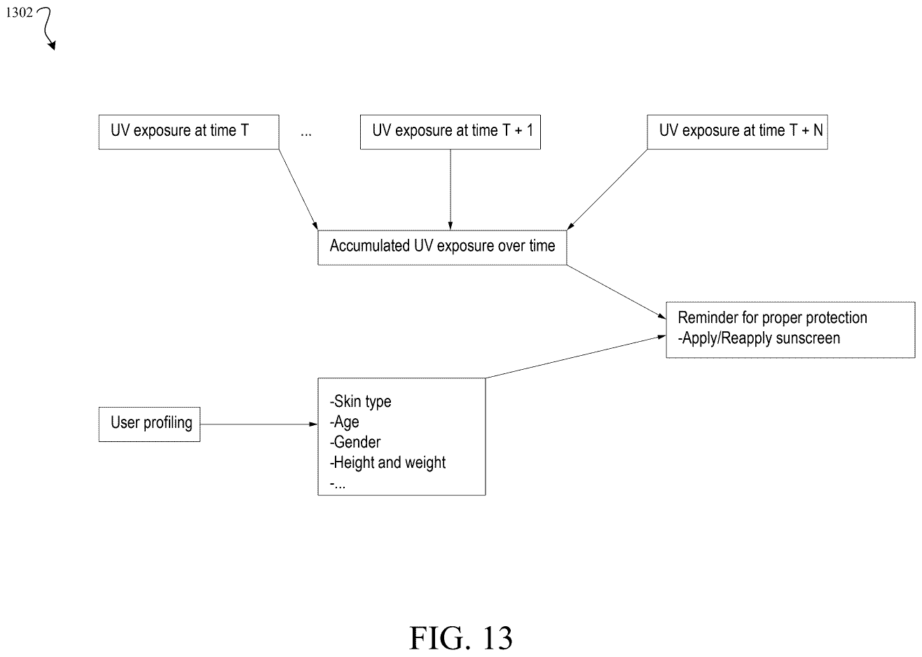

FIG. 13 is an illustration of an example process/technique 1302 to estimate an accumulated UV exposure over time. FIG. 13 shows how to calculate the accumulated UV exposure over time.

Pseudo code for EltaMD's server to push personalized content to the user follows.

EltaMD registers one official account. After the account is approved, EltaMD may get its own “APPLICATION ID” and “APPLICATION SECRET”.

One user subscribes to EltaMD's official account and consent to share location information with EltaMD. EltaMD can then estimate the user's UV exposure as the following pseudo-code.

```
  const axios = require(′axios′);
  // Function to get the current UV index based on latitude and longitude
  async function getUVIndex(latitude, longitude) {
    try {
      const apiKey = ′your_openuv_api_key′;
      const apiUrl =
‘https://api.openuv.io/api/v1/uv?lat=${latitude}&lng=${longitude}&appid=${apiKey}‘;
      const response = await axios.get(apiUrl);
      const uvIndex = response.data.result.uv;
      console.log(‘Current UV Index: ${uvIndex}‘);
      return uvIndex;
    } catch (error) {
      console.error(′Error fetching UV index:′, error);
      throw error;
    }
  }
  // Example usage
  const latitude = 40.7128; // Example latitude (New York City)
  const longitude = −74.0060; // Example longitude (New York City)
  getUVIndex(latitude, longitude)
    .then(uvIndex => {
      // Handle UV index
    })
    .catch(error => {
      // Handle error
    });
```

One user subscribes to EltaMA's official account. EltaMD can then get the user's “USER_OPENID”. EltaMD can push personalized information (e.g., personalized UV exposure information and recommendations etc.) to the user through this “USER_OPENID”.

```
  // Obtain access token using EltaMD's AppID and AppSecret
  String accessToken = getAccessToken(APPLICATION_ID,
APPLICATION_SECRET);
  // Sample JSON payload for customized subscription content
  JSONObject payload = new JSONObject( );
  // User's openID, once user subscribes EltaMD's official account
  // EltaMD would know this user's OPENID
  payload.put(“touser”, USER_OPENID); // User's OpenID
  payload.put(“template_id”, “your_template_id”); // Template ID for customized
content
  payload.put(“data”, new JSONObject( )); // Customized data for subscription content
  // Send customized subscription content using POST request
  OkHttpClient client = new OkHttpClient( );
  RequestBody body = RequestBody.create(MediaType.parse(“application/json”),
payload.toString( ));
  Request request = new Request.Builder( )
      .url(WECHAT_API_URL + accessToken)
      .post(body)
      .build( );
  try {
    Response response = client.newCall(request).execute( );
    System.out.println(response.body( ).string( ));
  } catch (IOException s) {
    e.printStackTrace( );
  }
```

Ultraviolet (UV) radiation is emitted by the sun and artificial sources, such as tanning beds. While it has some benefits for people, including the creation of Vitamin D, it also can cause health risks. It can cause premature aging of the skin and signs of sun damage such as wrinkles, leathery skin, liver spots, actinic keratosis, solar elastosis, and/or skin cancers.

The sun emits three different types of ultraviolet (UV) radiation. UVA: UVA comprises up to 95% of the light that reaches our skin. Prolonged and/or cumulative UVA exposure damages the collagen fibers in your skin, which contributes to signs of aging: wrinkles, age spots and loss of elasticity.

UVB: UVB makes up the rest of UV radiation that reaches the surface. UVB rays are the primary cause of sunburn. UVB exposure also accelerates skin aging, suppresses some immune functions and contributes to the development of skin cancer. UVC: The Earth's atmosphere absorbs UVC, so we only need to worry about UVA and UVB. Whether you are hiking, biking, swimming, and/or lounging this summer, take precautions are advisable against UV exposure such as with sunscreen, hats and sunglasses.

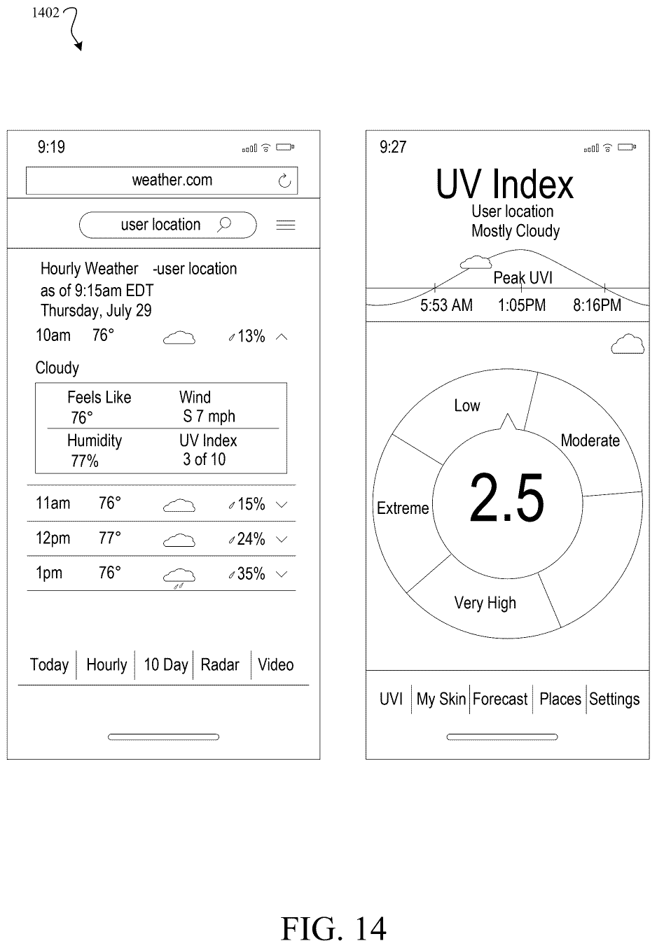

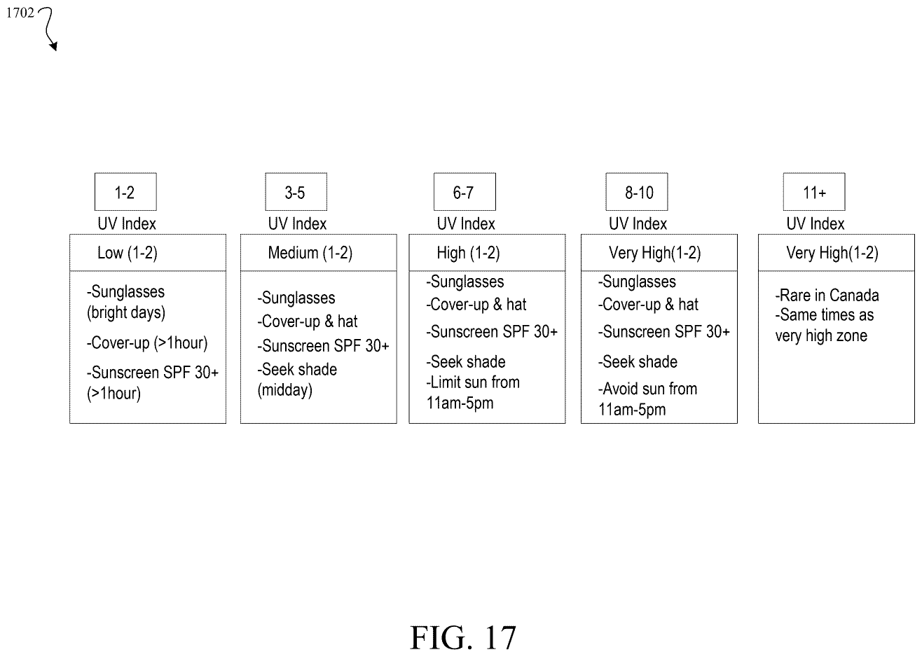

Before taking actions to protect yourself from prolonged UV exposure, it may be useful to know the UV exposure risks that a user may already have or may be going to have. Measuring UV exposure starts with tracking the UV index scale. One of the easiest ways to monitor sun exposure is looking at the daily UV index from smartphone weather Applications or some specialized UV tracking Applications. FIG. 14 is an example of a daily UV index report 1402 from smartphone-based weather Applications and/or specialized UV tracking Applications. FIG. 17 is an illustration of an example sun exposure presentation 1702 via a daily UV index per an electronic device/smartphone weather Application.

Such Applications estimate the UV index of the region, no matter if the user is indoors or outdoors, under the shade, or directly under the sun-which presents inaccuracies in real UV exposure determinations for the user.

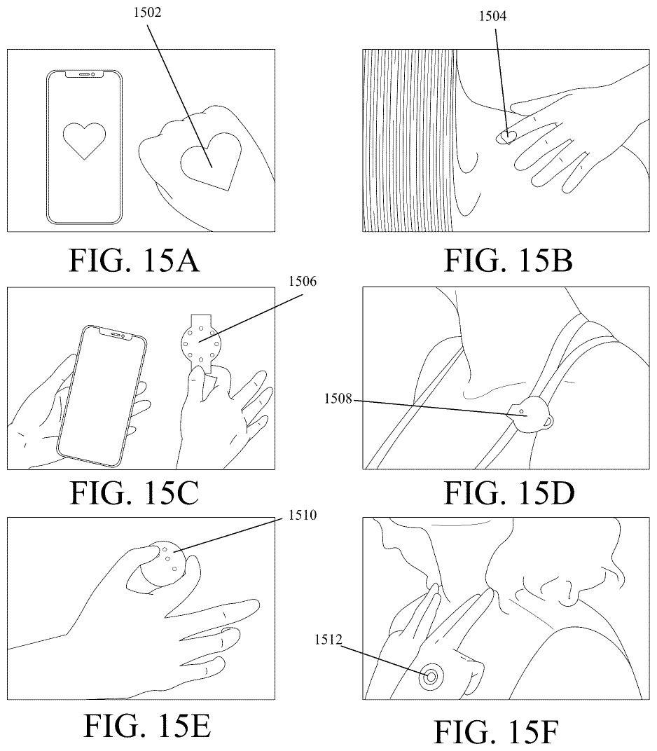

Wearable UV-tracking devices may be more accurate than relying on such UV index Applications. FIG. 15A to FIG. 15F are example illustrations of wearable UV-tracking devices 1502 to 1512. For example, L'Oréal' UV Patch and UV Sense, QSun, Shade, and/or Logic UV, etc., may provide such wearable UV-tracking devices.

Disclosed herein are techniques/devices/systems/methods of using an electronic device/smartphone Application without a wearable device, which can track the UV exposure more accurately than those weather Applications and UV Applications.

Existing weather Applications and/or specialized UV Applications may (e.g., mainly) get the UV index based on GPS location. However, actual UV exposure to the user greatly depends on the user's surrounding environment. For example, a user inside a building may receive 5-10 times less UV radiation than the UV index indicates. A user on a beach may receive higher UV radiation since the sand and/or water may reflect up the radiation.

One or more techniques described herein may (e.g., first) get the UV index value similar to the weather Applications and/or other existing UV Applications. As described with respect to FIG. 12, techniques may then calculate a multiplier based on the results of scene detection, indoors/outdoors, and/or activity recognition. With the UV index and/or the multiplier, the actual UV exposure may be estimated.

As described with respect to FIG. 13, perhaps once the techniques/Applications described herein determines an actual UV exposure at any given time, one or more user's exposure may be tracked periodically over time with certain intervals, such as every 3 minutes, among other intervals, to get the accumulated UV exposure over time. If the accumulated UV exposure may meet or exceed a certain threshold, perhaps based on a user's profile, the user may be reminded to take protective measures, such as applying/re-applying sunscreen to avoid the risks.

A UV Index in the region may be obtained. Techniques/Applications may get UV Index using OpenWeatherMap API services, using OpenWeatherMap that uses data from NOAA, using Met Office, using ECMWF, and/or using Environmental Canada.

Multipliers may be made on the UV Index for Scene Detection. Techniques/Applications may determine the type of environment the user is in. Possible environments include beaches, mountains, cities, and forests, etc. Techniques may use Geoapify Maps API to determine environment based on GPS location.

GPS signal strength may be determined. Since the GPS position may be determined via many satellites, there might not be “one” signal strength. For signal strength, the GpsStatus may be obtained via LocationManager.getGpsStatus( ) that may provide a list of satellites via getSatellites( ), and one or more, or each one, of those has a signal-to-noise ratio (getSnr( ). For GPS accuracy, Location.getAccuracy( ) may be used.

Indoor/outdoor detection may be conducted. In order to determine whether a user may be exposed to the sun or not, techniques/Applications may use the strength of the GPS signal from satellites. A weak signal may imply that the mobile device is indoors. A strong signal may imply that the device is outdoors.

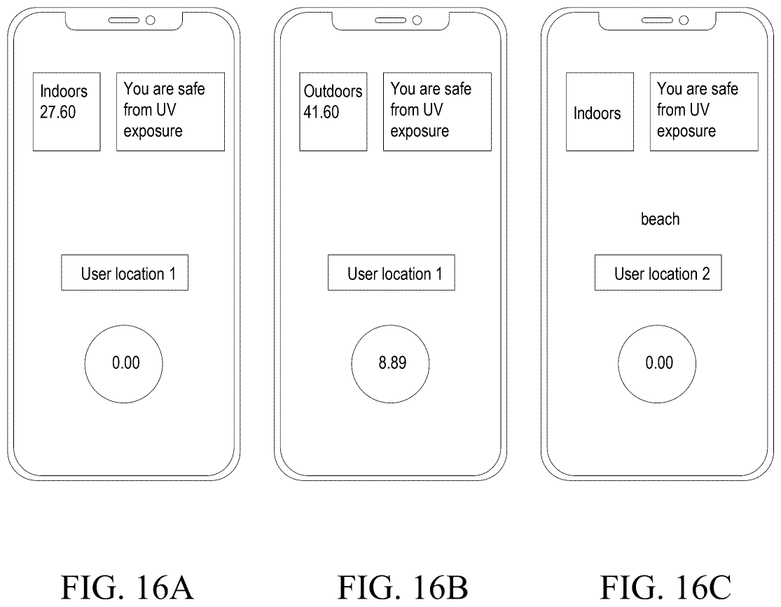

FIG. 16A to FIG. 16C are example illustrations of electronic device/smartphone indoor/outdoor detections/determinations and/or scene detections/determinations.

Techniques/Applications may rely on GPS signal strength to determine whether device is indoor or outdoor. Certain carrier-to-noise-density ratio signal values may indicate whether or not device is indoor or outdoor.

There may be several methods of determining the UV exposure of a user. The first is to use the single satellite that is closest to the sun. The second is to use all satellites available at a given time. Also, the accuracy of using the three satellites closest to the sun may be used.

Example results for each method are indicated below. The percentages indicated how many times the Application was able to correctly determine the correct environment of the user.

TABLE 1
Single Satellite

| | Indoors | Outdoor |
|---|---|---|
| Exposed | N/A | 75% |
| Not Exposed | 100% | 90% |

TABLE 2
All Satellites

| | Indoors | Outdoor |
|---|---|---|
| Exposed | N/A | 100% |
| Not Exposed | 100% | 14% |

TABLE 3
Three Satellites

| | Indoors | Outdoor |
|---|---|---|
| Exposed | N/A | 70% |
| Not Exposed | 95% | 95% |

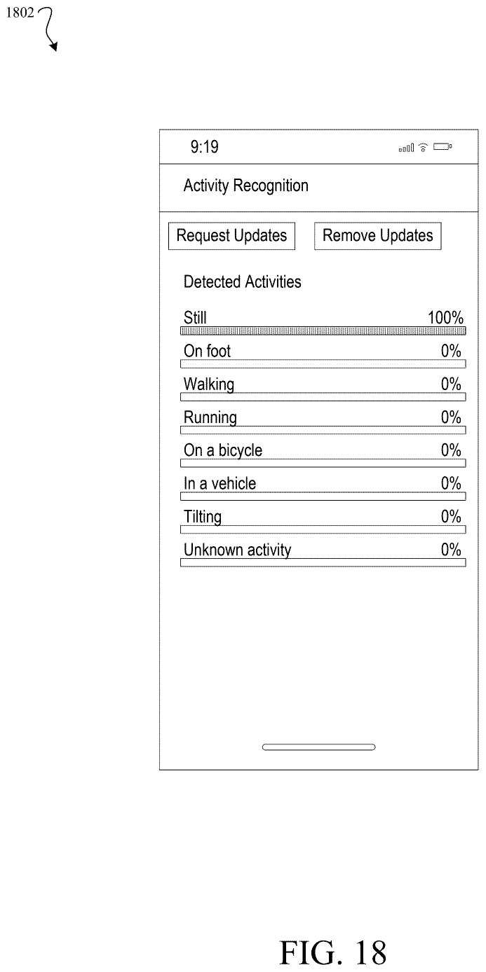

UV Exposure Estimation may be determined. Techniques/Applications may take the UV Index and may calculate the estimated UV exposure. The estimated exposure may be based on UV Index, the scene, and/or the activity. In one or more scenarios, one or more, or each type, of scene and/or activity may be assigned a reflection factor that may be multiplied as illustrated in FIG. 12, for example by the UV index, among one or more other factors/constants/variables, etc. One or more estimates may be compared with a UV detector. FIG. 18 is an illustration of an example of tracking GPS location changes/movements 1802 of an electronic device/smartphone to estimate an electronic device user's activity.

Storing Data and/or generating alerts may be done. Data of a user location and/or UV exposure may be stored in a SQLite database. This information may be used to determine the average UV exposure of the user in a given timeframe. Techniques/Applications may display an alert if the user has been exposed to large amounts of UV for a long period of time.

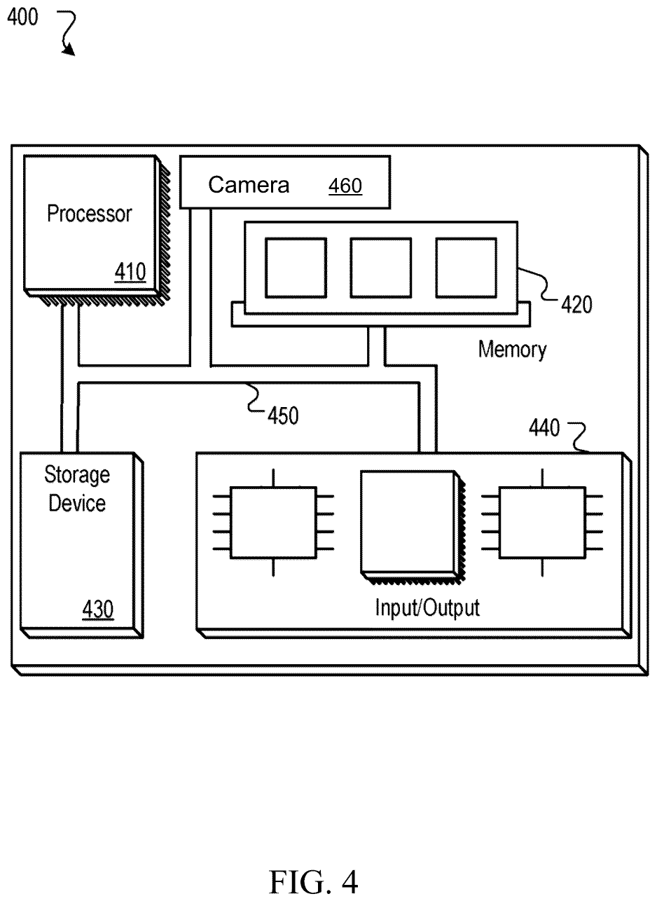

FIG. 4 is a block diagram of a hardware configuration of an example device that may function as a process control device/logic controller, such as the any of the devices of the Health and Environmental Exposure and Notification System 100 of FIG. 1, among other devices such as 140a-140i, and any of the devices 110a-110d, for example. The hardware configuration 400 may be operable to facilitate delivery of information from an internal server of a device. The hardware configuration 400 can include a processor 410, a memory 420, a storage device 430, and/or an input/output device 440. One or more of the components 410, 420, 430, and 440 can, for example, be interconnected using a system bus 450. The processor 410 can process instructions for execution within the hardware configuration 400. The processor 410 can be a single-threaded processor or the processor 410 can be a multi-threaded processor. The processor 410 can be a single-core processor or the processor 410 can be a multi-core processor. The processor 410 can be capable of processing instructions stored in the memory 420 and/or on the storage device 430. The processor 410 may be CPU, GPU, a hardware decoder(s) (e.g., for JPEG hardware decoder).

The memory 420 can store information within the hardware configuration 400. The memory 420 can be a computer-readable medium (CRM), for example, a non-transitory CRM. The memory 420 can be a volatile memory unit, and/or can be a non-volatile memory unit.

The storage device 430 can be capable of providing mass storage for the hardware configuration 400. The storage device 430 can be a computer-readable medium (CRM), for example, a non-transitory CRM. The storage device 430 can, for example, include a hard disk device, an optical disk device, flash memory and/or some other large capacity storage device. The storage device 430 can be a device external to the hardware configuration 400.

The input/output device 440 may provide input/output operations for the hardware configuration 400. The input/output device 440 (e.g., a transceiver device) can include one or more of a network interface device (e.g., an Ethernet card), a serial communication device (e.g., an RS-232 port), one or more universal serial bus (USB) interfaces (e.g., a USB 2.0 port) and/or a wireless interface device (e.g., an 802.11 card). The input/output device can include driver devices configured to send communications to, and/or receive communications from one or more networks (e.g., Health and Environmental Exposure and Notification Communication Network 130 of FIG. 1). The input/output device 400 may be in communication with one or more input/output modules (not shown) that may be proximate to the hardware configuration 400 and/or may be remote from the hardware configuration 400. The one or more output modules may provide input/output functionality in the digital signal form, discrete signal form, TTL form, analog signal form, serial communication protocol, fieldbus protocol communication and/or other open or proprietary communication protocol, and/or the like.

The camera device 460 may provide digital video input/output capability for the hardware configuration 400. The camera device 460 may communicate with any of the elements of the hardware configuration 400, perhaps for example via system bus 450. The camera device 460 may capture digital images and/or may scan images of various kinds, such as Universal Product Code (UPC) codes and/or Quick Response (QR) codes, for example, among other images as described herein. In one or more scenarios, the camera device 460 may be the same and/or substantially similar to any of the other camera devices described herein.

The camera device 460 may include at least one microphone device and/or at least one speaker device (not shown). The input/output of the camera device 460 may include audio signals/packets/components, perhaps for example separate/separable from, or in some (e.g., separable) combination with, the video signals/packets/components the camera device 460.

The camera device 460 may also detect the presence of one or more subjects that may be proximate to the camera device 460 and/or may be in the same general space (e.g., the same room, feeding area, etc.) as the camera device 460. The camera device 460 may gauge a general activity level (e.g., high activity, medium activity, and/or low activity) of one or more subjects that may be detected by the camera device 460. The camera device 460 may detect one or more general characteristics (e.g., height, body shape, skin color, pulse, heart rate, breathing count, etc.) of the one or more subjects detected by the camera device 460. The camera device 460 may be configured to recognize one or more specific subjects, for example. The camera device 460 may be configured to perform the one or more health and environmental exposure and notification techniques disclosed herein.

The camera device 460 may be in wired and/or wireless communication with the hardware configuration 400. In one or more scenarios, the camera device 460 may be external to the hardware configuration 400. In one or more scenarios, the camera device 460 may be internal to the hardware configuration 400.

In view of FIG. 1 to FIG. 18, one or more techniques, processes, systems, devices for providing health and/or environmental exposure tracking and/or notifications are described herein. A system for providing health and/or environmental exposure tracking and/or notifications may comprise at least a first sensor that may be configured to detect at least one of: a health parameter, or an environmental exposure parameter, corresponding to at least a first user. The system may comprise an electronic device. The electronic device may comprise a memory, a display, and/or a transceiver. The transceiver may be configured to communicate with at least one of: the first sensor, or a second processor. The first processor may be configured to configure at least one social media application on the electronic device. The first processor may be configured to receive, from the second processor, processed data corresponding to the at least one of: the health parameter, or the environmental exposure parameter, for presentation via the at least one social media application configured on the electronic device. The first processor may be configured to present the processed data corresponding to the at least one of: the health parameter, or the environmental exposure parameter, via one or more user-initiated sessions of the at least one social media application.

A system for providing health and/or environmental exposure tracking and/or notifications may comprise a processing device comprising at least a first processor. The first processor may be configured to receive, from a location service, location data corresponding to a location of an electronic device. The first processor may be configured to receive, from an environmental information service, an environmental exposure parameter associated with the location of the electronic device. The first processor may be configured to select at least one social media application configured on the electronic device. The first processor may be configured to generate an exposure data based on the environmental exposure parameter and the location data. The first processor may be configured to process the exposure data for presentation via the at least one social media application. The first processor may be configured to provide, to the electronic device, the processed exposure data for presentation via the at least one social media application.

The electronic device may comprise a memory, a display, and/or a transceiver. The transceiver may be configured to communicate with at least one of: the location service, the environmental information service, or the first processor. The electronic device may comprise a second processor. The second processor configured may be configured to configure the at least one social media application on the electronic device. The second processor may be configured to receive, from the first processor, the processed exposure data for presentation via the at least one social media application. The second processor may be configured to present the processed exposure data for presentation via the at least one social media application via one or more user-initiated sessions of the at least one social media application.

An electronic device for providing health and/or environmental exposure tracking and/or notifications may comprise a memory, a display, and/or a transceiver. The transceiver may be configured to communicate with one or more global position system (GPS) indicator devices and/or to communicate with an environmental information service. The electronic device may comprise a processor. The processor may be configured to determine, from the one or more global position system (GPS) indicator devices, a location of the electronic device. The processor may be configured to determine, from the environmental information service, an ultraviolet (UV) light index based on the location of the electronic device. The processor may be configured to determine, from the one or more global position system (GPS) indicator devices, movement data corresponding to the electronic device. The processor may be configured to determine at least one scene corresponding to the location of the electronic device. The processor may be configured to determine an activity type corresponding to the movement data. The processor may be configured to assign at least one multiplier to the scene and the activity type. The processor may be configured to determine a UV exposure corresponding to the location of the electronic device based, at least in part, on: a multiplied scene, a multiplied activity type, and the UV light index. The processor may be configured to present the UV exposure corresponding to the electronic device via the display of the electronic device.

A method for providing health and/or environmental exposure tracking and/or notifications may comprise detecting, by at least a first sensor, at least one of: a health parameter, or an environmental exposure parameter, corresponding to at least a first user. The method may comprise communicating, by a transceiver, with at least one of: the first sensor, or a second processor. The method may comprise configuring, by a first processor, at least one social media application on an electronic device. The method may comprise receiving, by the first processor from the second processor, processed data corresponding to the at least one of: the health parameter, or the environmental exposure parameter, for presentation via the at least one social media application configured on the electronic device. The method may comprise presenting the processed data corresponding to the at least one of: the health parameter, or the environmental exposure parameter, via one or more user-initiated sessions of the at least one social media application.

A method for providing health and/or environmental exposure tracking and/or notifications may comprise receiving, by a first processor from a location service, location data corresponding to a location of an electronic device. The method may comprise receiving, by the first processor from an environmental information service, an environmental exposure parameter associated with the location of the electronic device. The method may comprise selecting, by the first processor, at least one social media application configured on an electronic device. The method may comprise generating, by the first processor, an exposure data based on the environmental exposure parameter and the location data. The method may comprise processing, by the first processor, the exposure data for presentation via the at least one social media application. The method may comprise providing, by the first processor to the electronic device, the processed exposure data for presentation via the at least one social media application. The method may comprise communicating, by a transceiver, with at least one of: the location service, the environmental information service, or the first processor. The method may comprise configuring, by a second processor, the at least one social media application on the electronic device. The method may comprise receiving, by the second processor from the first processor, the processed exposure data for presentation via the at least one social media application. The method may comprise presenting, by the second processor, the processed exposure data for presentation via the at least one social media application via one or more user-initiated sessions of the at least one social media application.

A method performed by an electronic device for providing health and/or environmental exposure tracking and/or notifications may comprise communicating, by a transceiver, with one or more global position system (GPS) indicator devices. The method may comprise communicating, by the transceiver, with an environmental information service. The method may comprise determining, by a processor from the one or more global position system (GPS) indicator devices, a location of the electronic device. The method may comprise determining, by the processor from the environmental information service, an ultraviolet (UV) light index based on the location of the electronic device. The method may comprise determining, by the processor from the one or more global position system (GPS) indicator devices, movement data corresponding to the electronic device. The method may comprise determining, by the processor, at least one scene corresponding to the location of the electronic device. The method may comprise determining, by the processor, an activity type corresponding to the movement data. The method may comprise assigning, by the processor, at least one multiplier to the scene and the activity type. The method may comprise determining, by the processor, a UV exposure corresponding to the location of the electronic device based, at least in part, on: a multiplied scene, a multiplied activity type, and the UV light index. The method may comprise presenting, by the processor, the UV exposure corresponding to the location of the electronic device via a display of the electronic device.

The subject matter of this disclosure, and components thereof, can be realized by instructions that upon execution cause one or more processing devices to carry out the processes and/or functions described herein. Such instructions can, for example, comprise interpreted instructions, such as script instructions, e.g., JavaScript or ECMAScript instructions, or executable code, and/or other instructions stored in a computer readable medium.

Implementations of the subject matter and/or the functional operations described in this specification and/or the accompanying figures can be provided in digital electronic circuitry, in computer software, firmware, and/or hardware, including the structures disclosed in this specification and their structural equivalents, and/or in combinations of one or more of them. The subject matter described in this specification can be implemented as one or more computer program products, e.g., one or more modules of computer program instructions encoded on a tangible program carrier for execution by, and/or to control the operation of, data processing apparatus.

A computer program (also known as a program, software, software application, script, or code) can be written in any form of programming language, including compiled or interpreted languages, and/or declarative or procedural languages. It can be deployed in any form, including as a stand-alone program or as a module, component, subroutine, and/or other unit suitable for use in a computing environment. A computer program may or might not correspond to a file in a file system. A program can be stored in a portion of a file that holds other programs and/or data (e.g., one or more scripts stored in a markup language document), in a single file dedicated to the program in question, and/or in multiple coordinated files (e.g., files that store one or more modules, sub programs, or portions of code). A computer program can be deployed to be executed on one computer or on multiple computers that may be located at one site or distributed across multiple sites and/or interconnected by a communication network.

The processes and/or logic flows described in this specification and/or in the accompanying figures may be performed by one or more programmable processors executing one or more computer programs to perform functions by operating on input data and/or generating output, thereby tying the process to a particular machine (e.g., a machine programmed to perform the processes described herein). The processes and/or logic flows can also be performed by, and apparatus can also be implemented as, special purpose logic circuitry, e.g., an FPGA (field programmable gate array) and/or an ASIC (application specific integrated circuit).

Computer readable media suitable for storing computer program instructions and/or data may include all forms of non-volatile memory, media and memory devices, including by way of example semiconductor memory devices (e.g., EPROM, EEPROM, and/or flash memory devices); magnetic disks (e.g., internal hard disks or removable disks); magneto optical disks; and/or CD ROM and DVD ROM disks. The processor and/or the memory can be supplemented by, or incorporated in, special purpose logic circuitry.

While this specification and the accompanying figures contain many specific implementation details, these should not be construed as limitations on the scope of any invention and/or of what may be claimed, but rather as descriptions of features that may be specific to described example implementations. Certain features that are described in this specification in the context of separate implementations can also be implemented in combination in perhaps one implementation. Various features that are described in the context of perhaps one implementation can also be implemented in multiple combinations separately or in any suitable sub-combination. Although features may be described above as acting in certain combinations and/or perhaps even (e.g., initially) claimed as such, one or more features from a claimed combination can in some cases be excised from the combination. The claimed combination may be directed to a sub-combination and/or variation of a sub-combination.

While operations may be depicted in the drawings in an order, this should not be understood as requiring that such operations be performed in the particular order shown and/or in sequential order, and/or that all illustrated operations be performed, to achieve useful outcomes. The described program components and/or systems can generally be integrated together in a single software product and/or packaged into multiple software products.

Examples of the subject matter described in this specification have been described. The actions recited in the claims can be performed in a different order and still achieve useful outcomes, unless expressly noted otherwise. For example, the processes depicted in the accompanying figures do not require the particular order shown, and/or sequential order, to achieve useful outcomes. Multitasking and parallel processing may be advantageous in one or more scenarios.

While the present disclosure has been illustrated and described in detail in the drawings and foregoing description, the same is to be considered as illustrative and not restrictive in character, it being understood that only certain examples have been shown and described, and that all changes and modifications that come within the spirit of the present disclosure are desired to be protected.

### Exemplary Claims

Exemplary claim 1. A system for providing health and/or environmental exposure tracking and/or notifications, the system comprising: at least a first sensor configured to detect at least one of: a health parameter, or an environmental exposure parameter, corresponding to at least a first user; and an electronic device, comprising: a memory; a display; a transceiver, the transceiver configured to communicate with at least one of: the first sensor, or a second processor; and a first processor, the first processor configured at least to: configure at least one social media application on the electronic device; receive, from the second processor, processed data corresponding to the at least one of: the health parameter, or the environmental exposure parameter, for presentation via the at least one social media application configured on the electronic device; and present the processed data corresponding to the at least one of: the health parameter, or the environmental exposure parameter, via one or more user-initiated sessions of the at least one social media application.
Exemplary claim 2. The system of Exemplary claim 1, further comprising the second processor, the second processor configured at least to: receive, from the at least one sensor, data corresponding to at least one of: the health parameter, or the environmental exposure parameter; select the at least one social media application configured on the electronic device; process the data corresponding to the at least one of: the health parameter, or the environmental exposure parameter for presentation via the at least one social media application; and provide, to the electronic device, the processed data corresponding to the at least one of: the health parameter, or the environmental exposure parameter, for presentation via the at least one social media application.
Exemplary claim 3. The system of Exemplary claim 2, wherein the second processor is in communication with the transceiver via one or more wireless communication protocols.
Exemplary claim 4. The system of any one of Exemplary claims 2 to 3, wherein the second processor is part of one or more cloud-based processing devices.
Exemplary claim 5. The system of any of Exemplary claim 1 to Exemplary claim 4, wherein the at least one sensor is one or more of: a toothbrush, a heart-rate sensor, or an ultraviolent (UV) light sensor.
Exemplary claim 6. The system of any of Exemplary claim 2 to Exemplary claim 5, wherein the second processor is further configured such that the data corresponding to the at least one of: the health parameter, or the environmental exposure parameter, is received via direct communication from the at least one sensor.
Exemplary claim 7. The system of any of Exemplary claim 2 to Exemplary claim 5, wherein the second processor is further configured such that the data corresponding to the at least one of: the health parameter, or the environmental exposure parameter, is received from the at least one sensor via the transceiver.
Exemplary claim 8. The system of any of Exemplary claim 1 to Exemplary claim 7, wherein the at least one social media application is a first social media application, the first processor being further configured to associate a second or more social media applications with the electronic device.
Exemplary claim 9. The system of any of Exemplary claim 2 to Exemplary claim 8, wherein the first processor is further configured to: generate a real-time notification based on processed data corresponding to the at least one of: the health parameter, or the environmental exposure parameter, for presentation via the at least one social media application configured on the electronic device; and cause the real-time notification to be displayed on the display of the electronic device.
Exemplary claim 10. The system of any of Exemplary claim 2 to Exemplary claim 9, wherein the second processor is further configured to process the data corresponding to the at least one of: the health parameter, or the environmental exposure parameter, for presentation via the at least one social media application via at least one application programming interface (API) compatible with the at least one social media application.
Exemplary claim 11. A system for providing health and/or environmental exposure tracking and/or notifications, the system comprising: a processing device comprising at least a first processor, the first processor configured at least to: receive, from a location service, location data corresponding to a location of an electronic device; receive, from an environmental information service, an environmental exposure parameter associated with the location of the electronic device; select at least one social media application configured on the electronic device; generate an exposure data based on the environmental exposure parameter and the location data; process the exposure data for presentation via the at least one social media application; and provide, to the electronic device, the processed exposure data for presentation via the at least one social media application; and the electronic device, comprising: a memory; a display; a transceiver, the transceiver configured to communicate with at least one of: the location service, the environmental information service, or the first processor; and a second processor, the second processor configured at least to: configure the at least one social media application on the electronic device; receive, from the first processor, the processed exposure data for presentation via the at least one social media application; and present the processed exposure data for presentation via the at least one social media application via one or more user-initiated sessions of the at least one social media application.
Exemplary claim 12. The system of Exemplary claim 11, wherein the processing device comprises one or more processing cloud-based devices.
Exemplary claim 13. The system of any of Exemplary claim 11 or Exemplary claim 12, wherein at least one of: the location service, or the environmental information service, is a cloud-based service.
Exemplary claim 14. The system of any of Exemplary claim 11 to Exemplary claim 13, wherein the environmental exposure parameter is at least one of: an ultra-violet (UV) light level, a humidity level, a temperature value, a wind direction, or a wind speed.
Exemplary claim 15. The system of any of Exemplary claim 11 to Exemplary claim 14, wherein the exposure data indicates a quantification of environmental exposure at the location of the electronic device.
Exemplary claim 16. The system of Exemplary claim 15, wherein the first processor is further configured to determine the quantification of environmental exposure at the location of the electronic device over a period of time.
Exemplary claim 17. The system of any of Exemplary claim 15 to Exemplary claim 16, wherein the first processor is further configured to determine if the quantification of environmental exposure at the location of the electronic device meets, or exceeds, a predetermined threshold.
Exemplary claim 18. The system of any of Exemplary claim 11 to Exemplary claim 17, wherein the first processor is in communication with the at least one of: the location service, the environmental information service, or the first processor via one or more wireless communication protocols.
Exemplary claim 19. The system of any claim 11 to Exemplary claim 18, wherein the second processor is further configured to: generate a real-time notification based on the processed exposure data; and cause the real-time notification to be displayed on the display of the electronic device.
Exemplary claim 20. The system of any of Exemplary claim 11 to Exemplary claim 19, wherein the first processor is further configured to process the exposure data for presentation via the at least one social media application via at least one application programming interface (API) compatible with the at least one social media application.
Exemplary claim 21. The system of any of Exemplary claims 11 to 20, wherein the electronic device is a first electronic device, the environmental exposure parameter is a first environmental exposure parameter, and the exposure data is a first exposure data, the first processor is further configured to: receive, from the location service, location data corresponding to a location of a second electronic device; receive, from the environmental information service, a second environmental exposure parameter associated with the location of the second electronic device; generate a second exposure data based on the second environmental exposure parameter and the location data of the second electronic device; process the second exposure data for presentation via the at least one social media application; and provide, to the first electronic device, the processed second exposure data for presentation via the at least one social media application.
Exemplary claim 22. The system of Exemplary claim 21, wherein the second processor is further configured to: receive, from the first processor, the processed second exposure data for presentation via the at least one social media application; and present the processed second exposure data for presentation via the at least one social media application via the one or more user-initiated sessions of the at least one social media application.
Exemplary claim 23. The system of any of Exemplary claim 21 to Exemplary claim 22, wherein the real-time notification is a first real-time notification, the second processor being further configured to: generate a second real-time notification based on the processed second exposure data; and cause the second real-time notification to be displayed on the display of the first electronic device.
Exemplary claim 24. The system of any of Exemplary claims 11 to 23, wherein the location service is at least one of: a third-party location indication service relative to the electronic device and/or the processing device, or a location indication service configured on the electronic device.
Exemplary claim 25. The system of Exemplary claim 24, wherein at least one of: the third-party location indication service, or the location indication service configured on the electronic device, utilizes Global Positioning System (GPS) signaling.
Exemplary claim 26. An electronic device for providing health and/or environmental exposure tracking and/or notifications, the electronic device comprising: a memory; a display; a transceiver, the transceiver configured at least to: communicate with one or more global position system (GPS) indicator devices; and communicate with an environmental information service; and a processor, the processor configured at least to: determine, from the one or more global position system (GPS) indicator devices, a location of the electronic device; determine, from the environmental information service, an ultraviolet (UV) light index based on the location of the electronic device; determine, from the one or more global position system (GPS) indicator devices, movement data corresponding to the electronic device; determine at least one scene corresponding to the location of the electronic device; determine an activity type corresponding to the movement data; assign at least one multiplier to the scene and the activity type; determine a UV exposure corresponding to the location of the electronic device based, at least in part, on: a multiplied scene, a multiplied activity type, and the UV light index; and present the UV exposure corresponding to the electronic device via the display of the electronic device.
Exemplary claim 27. The device of Exemplary claim 26, wherein the one or more GPS indicator devices are at least of: one or more GPS satellites, or one or more GPS terrestrial stations.
Exemplary claim 28. The device of any of Exemplary claim 26 or claim 27, wherein the scene is at least one of: a beach, a forest, a mountain scene, a city scene, or a rural scene.
Exemplary claim 29. The device of any of Exemplary claim 26 to Exemplary claim 28, wherein the activity type is at least one of: vehicular transit, walking, running, or bicycling.
Exemplary claim 30. The device of any of Exemplary claim 26 to Exemplary claim 29, wherein the transceiver is further configured to: communicate with a satellite-based terrestrial imaging service, the processor being further configured to: acquire at least one image corresponding to the location of the electronic device from the satellite-based terrestrial imaging service; determine a first enclosure status of the electronic device based on the at least one image; and assign at least one multiplier to the first enclosure status of the electronic device, wherein the processor is further configured such that the UV exposure corresponding to the location of the electronic device is based, at least in part, on a multiplied first enclosure status.
Exemplary claim 31. The device of Exemplary claim 30, wherein the first enclosure status is at least one of: an indoor status, an outdoor status, an outdoor beach status, an outdoor forest status, or an outdoor mountain status.
Exemplary claim 32. The device of any of Exemplary claim 26 to Exemplary claim 31, wherein the processor is further configured to: determine, from the one or more global position system (GPS) indicator devices, a GPS signal strength of the electronic device; determine a second enclosure status of the electronic device based on the GPS signal strength of the electronic device; and assign at least one multiplier to the second enclosure status of the electronic device, wherein the processor is further configured such that the UV exposure corresponding to the location of the electronic device is based, at least in part, on a multiplied second enclosure status.
Exemplary claim 33. The device of Exemplary claim 32, wherein the second enclosure status is at least one of: an indoor exposure status, or an outdoor exposure status.
Exemplary claim 34. The device of any of Exemplary claim 26 to Exemplary claim 33, wherein the processor is further configured to: determine a plurality of UV exposures corresponding to the location of the electronic device over a period of time; determine an accumulated UV exposure based on the plurality of UV exposures; determine a UV-based health recommendation for a user of the electronic device, based at least in part, on the accumulated UV exposure; and display the UV-based health recommendation on the display of the electronic device.
Exemplary claim 35. The system of Exemplary claim 34, wherein the processor is further configured to determine a user profile of a user of the electronic device, the user profile comprising at least one of: a skin type of the user, an age of the user, a gender of the user, a height of the user, or a weight of the user.
Exemplary claim 36. The device of Exemplary claim 35, wherein the processor is further configured to determine the UV-based health recommendation for the user of the electronic device, based at least in part, on the user profile.
Exemplary claim 37. The device of any of Exemplary claim 26 to Exemplary claim 36, wherein the transceiver is further configured to communicate with a location service, the processor being further configured to: provide the location of the electronic device to the location service, the location service providing the location of the electronic device to a health care advice service; and receive, from the health care advice service, health care advice data corresponding to the location of the electronic device and a user of the electronic device.
Exemplary claim 38. The device of Exemplary claim 37, wherein the processor is further configured to: process the health care advice data for presentation via at least one social media application configured on the electronic device; and present the processed health care advice data for presentation via the at least one social media application via one or more user-initiated sessions of the at least one social media application.
Exemplary claim 39. The device of Exemplary claim 38, wherein the processor is further configured to: generate a real-time notification based on the processed health care advice data; and cause the real-time notification to be displayed on the display of the electronic device.
Exemplary claim 40. The device of any of Exemplary claim 37 to Exemplary claim 39, wherein the location service is at least one of: a third-party location indication service relative to the electronic device, or a location indication service configured on the electronic device.
Exemplary claim 41. A method for providing health and/or environmental exposure tracking and/or notifications, the method comprising: detecting, by at least a first sensor, at least one of: a health parameter, or an environmental exposure parameter, corresponding to at least a first user; communicating, by a transceiver, with at least one of: the first sensor, or a second processor; configuring, by a first processor, at least one social media application on an electronic device; receiving, by the first processor from the second processor, processed data corresponding to the at least one of: the health parameter, or the environmental exposure parameter, for presentation via the at least one social media application configured on the electronic device; and presenting the processed data corresponding to the at least one of: the health parameter, or the environmental exposure parameter, via one or more user-initiated sessions of the at least one social media application.
Exemplary claim 42. The method of Exemplary claim 41, further comprising: receiving, by the second processor from the at least one sensor, data corresponding to at least one of: the health parameter, or the environmental exposure parameter; selecting, by the second processor, the at least one social media application configured on the electronic device; processing, by the second processor, the data corresponding to the at least one of: the health parameter, or the environmental exposure parameter for presentation via the at least one social media application; and providing, by the second processor to the electronic device, the processed data corresponding to the at least one of: the health parameter, or the environmental exposure parameter, for presentation via the at least one social media application.
Exemplary claim 43. The method of Exemplary claim 42, wherein the second processor is in communication with the transceiver via one or more wireless communication protocols.
Exemplary claim 44. The method of any of Exemplary claim 42 or claim 43, wherein the second processor is part of one or more cloud-based processing devices.
Exemplary claim 45. The method of any of Exemplary claim 41 to Exemplary claim 44, wherein the at least one sensor is one or more of: a toothbrush, a heart-rate sensor, or an ultraviolent (UV) light sensor.
Exemplary claim 46. The method of any of Exemplary claim 42 to Exemplary claim 45, wherein the second processor is further configured such that the data corresponding to the at least one of: the health parameter, or the environmental exposure parameter, is received via direct communication from the at least one sensor.
Exemplary claim 47. The method of any of Exemplary claim 42 to Exemplary claim 45, wherein the second processor is further configured such that the data corresponding to the at least one of: the health parameter, or the environmental exposure parameter, is received from the at least one sensor via the transceiver
Exemplary claim 48. The method of any of Exemplary claim 41 to Exemplary claim 47, wherein the at least one social media application is a first social media application, the first processor being further configured to associate a second or more social media applications with the electronic device.
Exemplary claim 49. The method of any of Exemplary claim 42 to Exemplary claim 48, further comprising: generating, by the first processor, a real-time notification based on processed data corresponding to the at least one of: the health parameter, or the environmental exposure parameter, for presentation via the at least one social media application configured on the electronic device; and causing, by the first processor, the real-time notification to be displayed on the display of the electronic device.
Exemplary claim 50. The method of any of Exemplary claim 42 to Exemplary claim 49, further comprising, processing, by the second processor, the data corresponding to the at least one of: the health parameter, or the environmental exposure parameter, for presentation via the at least one social media application via at least one application programming interface (API) compatible with the at least one social media application.
Exemplary claim 51. A method for providing health and/or environmental exposure tracking and/or notifications, the method comprising: receiving, by a first processor from a location service, location data corresponding to a location of an electronic device; receiving, by the first processor from an environmental information service, an environmental exposure parameter associated with the location of the electronic device; selecting, by the first processor, at least one social media application configured on an electronic device; generating, by the first processor, an exposure data based on the environmental exposure parameter and the location data; processing, by the first processor, the exposure data for presentation via the at least one social media application; providing, by the first processor to the electronic device, the processed exposure data for presentation via the at least one social media application; communicating, by a transceiver, with at least one of: the location service, the environmental information service, or the first processor; configuring, by a second processor, the at least one social media application on the electronic device; receiving, by the second processor from the first processor, the processed exposure data for presentation via the at least one social media application; and presenting, by the second processor, the processed exposure data for presentation via the at least one social media application via one or more user-initiated sessions of the at least one social media application.
Exemplary claim 52. The method of Exemplary claim 51, wherein the first processor comprises one or more cloud-based processing devices.
Exemplary claim 53. The method of any of Exemplary claim 51 or claim 52, wherein at least one of: the location service, or the environmental information service, is a cloud-based service.
Exemplary claim 54. The method of any of Exemplary claim 51 to Exemplary claim 53, wherein the environmental exposure parameter is at least one of: an ultra-violet (UV) light level, a humidity level, a temperature value, a wind direction, or a wind speed.
Exemplary claim 55. The method of any of Exemplary claim 51 to Exemplary claim 54, wherein the exposure data indicates a quantification of environmental exposure at the location of the electronic device.
Exemplary claim 56. The method of Exemplary claim 55, further comprising determining, by the first processor, the quantification of environmental exposure at the location of the electronic device over a period of time.
Exemplary claim 57. The method of any of Exemplary claim 55 to Exemplary claim 56, further comprising, by the first processor, the quantification of environmental exposure at the location of the electronic device meets, or exceeds, a predetermined threshold.
Exemplary claim 58. The method of any of Exemplary claim 51 to Exemplary claim 57, wherein the first processor is in communication with the at least one of: the location service, the environmental information service, or the first processor via one or more wireless communication protocols.
Exemplary claim 59. The method of any claim 51 to Exemplary claim 58, further comprising: generating, by the second processor, a real-time notification based on the processed exposure data; and causing, by the second processor, the real-time notification to be displayed on the display of the electronic device.
Exemplary claim 60. The method of any of Exemplary claim 51 to Exemplary claim 59, further comprising processing, by the first processor, the exposure data for presentation via the at least one social media application via at least one application programming interface (API) compatible with the at least one social media application.
Exemplary claim 61. The method of any of Exemplary claims 51 to 60, wherein the electronic device is a first electronic device, the environmental exposure parameter is a first environmental exposure parameter, and the exposure data is a first exposure data, the method further comprising: receiving, by the first processor from the location service, location data corresponding to a location of a second electronic device; receiving, by the first processor from the environmental information service, a second environmental exposure parameter associated with the location of the second electronic device; generating, by the first processor, a second exposure data based on the second environmental exposure parameter and the location data of the second electronic device; processing, by the first processor, the second exposure data for presentation via the at least one social media application; and providing, by the first processor to the first electronic device, the processed second exposure data for presentation via the at least one social media application.
Exemplary claim 62. The method of Exemplary claim 61, further comprising: receiving, by the second processor from the first processor, the processed second exposure data for presentation via the at least one social media application; and presenting, by the second processor, the processed second exposure data for presentation via the at least one social media application via the one or more user-initiated sessions of the at least one social media application.
Exemplary claim 63. The method of any of Exemplary claim 61 to Exemplary claim 62, wherein the real-time notification is a first real-time notification, the method further comprising: generating, by the second processor, a second real-time notification based on the processed second exposure data; and causing, by the second processor, the second real-time notification to be displayed on the display of the first electronic device.
Exemplary claim 64. The method of any of Exemplary claims 51 to 63, wherein the location service is at least one of: a third-party location indication service relative to the electronic device and/or the processing device, or a location indication service configured on the electronic device.
Exemplary claim 65. The method of Exemplary claim 64, wherein at least one of: the third-party location indication service, or the location indication service configured on the electronic device, utilizes Global Positioning System (GPS) signaling.
Exemplary claim 66. A method performed by an electronic device for providing health and/or environmental exposure tracking and/or notifications, the method comprising: communicating, by a transceiver, with one or more global position system (GPS) indicator devices; communicating, by the transceiver, with an environmental information service; determining, by a processor from the one or more global position system (GPS) indicator devices, a location of the electronic device; determining, by the processor from the environmental information service, an ultraviolet (UV) light index based on the location of the electronic device; determining, by the processor from the one or more global position system (GPS) indicator devices, movement data corresponding to the electronic device; determining, by the processor, at least one scene corresponding to the location of the electronic device; determining, by the processor, an activity type corresponding to the movement data; assigning, by the processor, at least one multiplier to the scene and the activity type; determining, by the processor, a UV exposure corresponding to the location of the electronic device based, at least in part, on: a multiplied scene, a multiplied activity type, and the UV light index; and presenting, by the processor, the UV exposure corresponding to the location of the electronic device via a display of the electronic device.
Exemplary claim 67. The method of Exemplary claim 66, wherein the one or more GPS indicator devices are at least of: one or more GPS satellites, or one or more GPS terrestrial stations.
Exemplary claim 68. The method of any of Exemplary claim 66 or claim 67, wherein the scene is at least one of: a beach, a forest, a mountain scene, a city scene, or a rural scene.
Exemplary claim 69. The method of any of Exemplary claim 66 to Exemplary claim 68, wherein the activity type is at least one of: vehicular transit, walking, running, or bicycling.
Exemplary claim 70. The method of any of Exemplary claim 66 to Exemplary claim 69, further comprising: communicating, by the transceiver, with a satellite-based terrestrial imaging service; acquiring, by the processor, at least one image corresponding to the location of the electronic device from the satellite-based terrestrial imaging service; determining, by the processor, a first enclosure status of the electronic device based on the at least one image; and assigning, by the processor, at least one multiplier to the first enclosure status of the electronic device, wherein the determining, by the processor, the UV exposure corresponding to the location of the electronic device is based, at least in part, on a multiplied first enclosure status.
Exemplary claim 71. The method of Exemplary claim 70, wherein the first enclosure status is at least one of: an indoor status, an outdoor status, an outdoor beach status, an outdoor forest status, or an outdoor mountain status.
Exemplary claim 72. The method of any of Exemplary claim 66 to Exemplary claim 71, further comprising: determining, by the processor from the one or more global position system (GPS) indicator devices, a GPS signal strength of the electronic device; determining, by the processor, a second enclosure status of the electronic device based on the GPS signal strength of the electronic device; and assigning, by the processor, at least one multiplier to the second enclosure status of the electronic device, wherein the determining, by the processor, the UV exposure corresponding to the location of the electronic device is based, at least in part, on a multiplied second enclosure status.
Exemplary claim 73. The method of Exemplary claim 72, wherein the second enclosure status is at least one of: an indoor exposure status, or an outdoor exposure status.
Exemplary claim 74. The method of any of Exemplary claim 66 to Exemplary claim 73, further comprising: determining, by the processor, a plurality of UV exposures corresponding to the location of the electronic device over a period of time; determining, by the processor, an accumulated UV exposure based on the plurality of UV exposures; determining, by the processor, a UV-based health recommendation for a user of the electronic device, based at least in part, on the accumulated UV exposure; and displaying, by the processor, the UV-based health recommendation on the display of the electronic device.
Exemplary claim 75. The method of Exemplary claim 74, further comprising determining, by the processor, a user profile of a user of the electronic device, the user profile comprising at least one of: a skin type of the user, an age of the user, a gender of the user, a height of the user, or a weight of the user.
Exemplary claim 76. The method of Exemplary claim 75, further comprising determining, by the processor, the UV-based health recommendation for the user of the electronic device, based at least in part, on the user profile.
Exemplary claim 77. The method of any of Exemplary claim 66 to Exemplary claim 76, further comprising: communicating, by the transceiver, with a location service; providing, by the processor, the location of the electronic device to the location service, the location service providing the location of the electronic device to a health care advice service; and receiving, by the processor from the health care advice service, health care advice data corresponding to the location of the electronic device and a user of the electronic device.
Exemplary claim 78. The method of Exemplary claim 77, further comprising: processing, by the processor, the health care advice data for presentation via at least one social media application configured on the electronic device; and presenting, by the processor, the processed health care advice data for presentation via the at least one social media application via one or more user-initiated sessions of the at least one social media application.
Exemplary claim 79. The method of Exemplary claim 78, further comprising: generating, by the processor, a real-time notification based on the processed health care advice data; and causing, by the processor, the real-time notification to be displayed on the display of the electronic device.
Exemplary claim 80. The method of any of Exemplary claim 77 to Exemplary claim 79, wherein the location service is at least one of: a third-party location indication service relative to the electronic device, or a location indication service configured on the electronic device.

What is claimed is:

1. A system for providing health and/or environmental exposure tracking and/or notifications, the system comprising:
at least a first sensor configured to detect at least one of: a health parameter, or an environmental exposure parameter, corresponding to at least a first user; and
an electronic device, comprising:
a memory;
a display;
a transceiver, the transceiver configured to communicate with at least one of: the first sensor, or a second processor; and
a first processor, the first processor configured at least to:
configure at least one social media application on the electronic device;
receive, from the second processor, processed data corresponding to the at least one of: the health parameter, or the environmental exposure parameter, for presentation via the at least one social media application configured on the electronic device; and
present the processed data corresponding to the at least one of: the health parameter, or the environmental exposure parameter, via one or more user-initiated sessions of the at least one social media application.

2. The system according to claim 1, further comprising the second processor, the second processor configured at least to:
receive, from the at least one sensor, data corresponding to at least one of: the health parameter, or the environmental exposure parameter;
select the at least one social media application configured on the electronic device;
process the data corresponding to the at least one of: the health parameter, or the environmental exposure parameter for presentation via the at least one social media application; and
provide, to the electronic device, the processed data corresponding to the at least one of: the health parameter, or the environmental exposure parameter, for presentation via the at least one social media application.

3. The system according to claim 2, wherein the second processor is in communication with the transceiver via one or more wireless communication protocols.

4. The system according to claim 2, wherein the second processor is part of one or more cloud-based processing devices.

5. The system according to claim 2, wherein the second processor is further configured such that the data corresponding to the at least one of: the health parameter, or the environmental exposure parameter, is received via direct communication from the at least one sensor.

6. The system according to claim 2, wherein the second processor is further configured such that the data corresponding to the at least one of: the health parameter, or the environmental exposure parameter, is received from the at least one sensor via the transceiver.

7. The system according to claim 2, wherein the first processor is further configured to:
generate a real-time notification based on processed data corresponding to the at least one of: the health parameter, or the environmental exposure parameter, for presentation via the at least one social media application configured on the electronic device; and
cause the real-time notification to be displayed on the display of the electronic device.

8. The system according to claim 2, wherein the second processor is further configured to process the data corresponding to the at least one of: the health parameter, or the environmental exposure parameter, for presentation via the at least one social media application via at least one application programming interface (API) compatible with the at least one social media application.

9. The system according to claim 1, wherein the at least one sensor is one or more of: a toothbrush, a heart-rate sensor, or an ultraviolent (UV) light sensor.

10. The system according to claim 1, wherein the at least one social media application is a first social media application, the first processor being further configured to associate a second or more social media applications with the electronic device.

11. A system for providing health and/or environmental exposure tracking and/or notifications, the system comprising:
a processing device comprising at least a first processor, the first processor configured at least to:
receive, from a location service, location data corresponding to a location of an electronic device;
receive, from an environmental information service, an environmental exposure parameter associated with the location of the electronic device;
select at least one social media application configured on the electronic device;
generate an exposure data based on the environmental exposure parameter and the location data;
process the exposure data for presentation via the at least one social media application; and
provide, to the electronic device, the processed exposure data for presentation via the at least one social media application; and
the electronic device, comprising:
a memory;
a display;
a transceiver, the transceiver configured to communicate with at least one of: the location service, the environmental information service, or the first processor; and
a second processor, the second processor configured at least to:
configure the at least one social media application on the electronic device;
receive, from the first processor, the processed exposure data for presentation via the at least one social media application; and
present the processed exposure data for presentation via the at least one social media application via one or more user-initiated sessions of the at least one social media application.

12. The system according to claim 11, wherein the environmental exposure parameter is at least one of: an ultra-violet (UV) light level, a humidity level, a temperature value, a wind direction, or a wind speed.

13. The system according to claim 11, wherein the exposure data indicates a quantification of environmental exposure at the location of the electronic device, wherein the first processor is further configured to determine the quantification of environmental exposure at the location of the electronic device over a period of time and determine if the quantification of environmental exposure at the location of the electronic device meets, or exceeds, a predetermined threshold.

14. The system according to claim 11, wherein the second processor is further configured to:
generate a real-time notification based on the processed exposure data; and
cause the real-time notification to be displayed on the display of the electronic device.

15. The system according to claim 11, wherein the electronic device is a first electronic device, the environmental exposure parameter is a first environmental exposure parameter, and the exposure data is a first exposure data, the first processor is further configured to:
receive, from the location service, location data corresponding to a location of a second electronic device;
receive, from the environmental information service, a second environmental exposure parameter associated with the location of the second electronic device;
generate a second exposure data based on the second environmental exposure parameter and the location data of the second electronic device;
process the second exposure data for presentation via the at least one social media application; and
provide, to the first electronic device, the processed second exposure data for presentation via the at least one social media application.

16. An electronic device for providing health and/or environmental exposure tracking and/or notifications, the electronic device comprising:
a memory;
a display;
a transceiver, the transceiver configured at least to:
communicate with one or more global position system (GPS) indicator devices; and
communicate with an environmental information service; and
a processor, the processor configured at least to:
determine, from the one or more global position system (GPS) indicator devices, a location of the electronic device;
determine, from the environmental information service, an ultraviolet (UV) light index based on the location of the electronic device;
determine, from the one or more global position system (GPS) indicator devices, movement data corresponding to the electronic device;
determine at least one scene corresponding to the location of the electronic device;
determine an activity type corresponding to the movement data;
assign at least one multiplier to the scene and the activity type;
determine a UV exposure corresponding to the location of the electronic device based, at least in part, on: a multiplied scene, a multiplied activity type, and the UV light index; and
present the UV exposure corresponding to the electronic device via the display of the electronic device.

17. The electronic device according to claim 16, wherein the one or more GPS indicator devices are at least of: one or more GPS satellites, or one or more GPS terrestrial stations, wherein the scene is at least one of: a beach, a forest, a mountain scene, a city scene, or a rural scene, and wherein the activity type is at least one of: vehicular transit, walking, running, or bicycling.

18. The electronic device according to claim 16, wherein the transceiver is further configured to: communicate with a satellite-based terrestrial imaging service, the processor being further configured to:
acquire at least one image corresponding to the location of the electronic device from the satellite-based terrestrial imaging service;
determine a first enclosure status of the electronic device based on the at least one image, wherein the first enclosure status is at least one of: an indoor status, an outdoor status, an outdoor beach status, an outdoor forest status, or an outdoor mountain status; and
assign at least one multiplier to the first enclosure status of the electronic device, wherein the processor is further configured such that the UV exposure corresponding to the location of the electronic device is based, at least in part, on a multiplied first enclosure status.

19. The electronic device according to claim 16, wherein the processor is further configured to:
determine, from the one or more global position system (GPS) indicator devices, a GPS signal strength of the electronic device;
determine a second enclosure status of the electronic device based on the GPS signal strength of the electronic device, wherein the second enclosure status is at least one of: an indoor exposure status, or an outdoor exposure status; and
assign at least one multiplier to the second enclosure status of the electronic device, wherein the processor is further configured such that the UV exposure corresponding to the location of the electronic device is based, at least in part, on a multiplied second enclosure status.

20. The electronic device according to claim 16, wherein the processor is further configured to:
determine a plurality of UV exposures corresponding to the location of the electronic device over a period of time;
determine an accumulated UV exposure based on the plurality of UV exposures;
determine a UV-based health recommendation for a user of the electronic device, based at least in part, on the accumulated UV exposure; and
display the UV-based health recommendation on the display of the electronic device;
determine a user profile of a user of the electronic device, the user profile comprising at least one of: a skin type of the user, an age of the user, a gender of the user, a height of the user, or a weight of the user; and
determine the UV-based health recommendation for the user of the electronic device, based at least in part, on the user profile.
